(19) United States
(12) Patent Application Publication
(10) Pub. No.: US 2022/0196667 A1
(43) Pub. Date: Jun. 23, 2022

(54) TUMOR MICROBIOME SIGNATURE AND THERAPEUTIC USE OF FECAL MICROBIOTA TRANSPLANTATION ON PANCREATIC CANCER PATIENTS

(71) Applicant: Board of Regents, The University of Texas System, Austin, TX (US)

(72) Inventors: Florencia McALLISTER, Houston, TX (US); Yu ZHANG, Houston, TX (US); Erick RIQUELME, Houston, TX (US)

(73) Assignee: Board of Regents, The University of Texas System, Austin, TX (US)

(21) Appl. No.: 17/600,596

(22) PCT Filed: Apr. 1, 2020
(86) PCT No.: PCT/US2020/026102

§ 371 Date: Sep. 30, 2021

Related U.S. Application Data
(60) Provisional application No. 62/904,323, filed on Sep. 23, 2019.
(60) Provisional application No. 62/827,530, filed on Apr. 1, 2019.

(51) Int. Cl. G01N 33/574; G01N 33/569; A61K 45/06

(52) U.S. Cl. CPC G01N 33/57438; A61K 45/06; G01N 33/56911

(57) ABSTRACT

Provided herein are methods of predicting whether a pancreatic cancer patient will be a short-term or long-term survivor based on their intra-tumoral microbiota. Also provided are methods of treating pancreatic cancer patients using fecal microbial transfer from long-term pancreatic cancer survivors as well as pharmaceutical compositions comprising fecal microbiota obtained from long-term pancreatic cancer survivors.

### REFERENCE TO RELATED APPLICATIONS

The present application claims the priority benefit of U.S. provisional application No. 62/904,323, filed Sep. 23, 2019, and U.S. provisional application No. 62/827,530, filed Apr. 1, 2019, the entire contents of each of which is incorporated herein by reference.

### BACKGROUND

### 1. Field

The present invention relates generally to the fields of medicine and oncology. More particularly, it concerns methods of predicting whether a pancreatic cancer patient will be a short-term or long-term survivor as well as methods of treating pancreatic cancer patients using fecal microbial transfer.

### 2. Description of Related Art

Pancreatic ductal adenocarcinoma (PDAC) is a disease of near uniform mortality (Hidalgo, 2010; Kamisawa et al., 2016; Miller et al., 2016). Most patients present with advanced stage disease and the prognosis is dismal, with a 5-year overall survival of 9% (Siegel et al., 2018). Even when patients can undergo surgical resection, the recurrence rate is very high and median overall survival varies between 24 to 30 months (Siegel et al., 2018). Despite this, a minor subset of patients survives more than 5-years post-surgery (Dal Molin et al., 2015; DeSantis et al., 2014). The factors that determine such enigmatic long-term survival are unknown. It is widely accepted that the cancer genomic landscape can be predictive of overall survival and response to therapy (Le et al., 2015; Ock et al., 2017; Vogelstein et al., 2013). However, analysis of stage-matched PDAC long term survivors has not demonstrated significant genomic differences compared to those patients with shorter survival (Dal Molin et al., 2015; Makohon-Moore et al., 2017). Recently, Balachandran et al. (2017) used an in silico prediction approach to demonstrate that PDAC tumors from long term survivors have high quantity and quality of neoantigens, and stronger infiltration and activation of CD8+ T cells. Interestingly, the neoantigens exhibited homology to infectious disease-derived peptides, suggesting a neoantigen molecular mimicry with microbial epitopes (Balachandran et al., 2017). These data suggest that microbial host factors, independent of the genomic composition of the tumor, may determine tumor behavior and patient outcomes.

The key role of the gut microbiota as a host factor mediating tumor responses to chemotherapy and immunotherapy in patients with melanoma and lung cancers has been recently highlighted (Gopalakrishnan et al., 2018; Matson et al., 2018; Routy et al., 2018). These studies suggest that gut bacteria influence the activation of the immune system, promoting cancer-associated inflammation, and ultimately affecting tumor responses to therapies. This information has allowed the stratification of patients into responders and non-responders using the microbiota composition as a predictive biomarker of response to immunotherapy.

Recently, Geller et al. (2017) reported the presence of Gammaproteobacteria (GP), besides other bacteria, in human PDAC. Importantly, they reported that GP is able to metabolize gemcitabine (2′,2′-difluorodeoxycytidine) into its inactive form (2′,2′-difluorodeoxyuridine), suggesting that the presence of this bacteria in PDAC may be responsible for the tumor resistance to gemcitabine. Additionally, Pushalkar et al. (2018) detected specific gut and tumor microbiome in murine models of PDAC, suggesting the existence of potential bacterial translocation from the intestinal tract into the peritumoral milieu. Bacterial ablation with antibiotics in a PDAC orthotopic mouse model reshapes the tumor microenvironment, inducing T-cell activation, improving immune surveillance, and increasing sensitivity to immunotherapy. Together, these data suggest that modulating the gut and/or tumor microbiome could emerge as a novel strategy to sensitize tumors to therapeutics.

Despite all this knowledge, the composition of the human PDAC microbiome that contributes favorably or adversely to the natural history of pancreatic cancer remains incompletely studied. This represents a significant unmet need, as most chemotherapeutic and immunotherapeutic agents that have proven efficacy in other malignancies have limited efficacy in PDAC (Garrido-Laguna and Hidalgo, 2015; Manji et al., 2017). Therefore, methods for using the microbiome signature to make prognostic decisions in PDAC patients are needed. Additionally, methods are needed for manipulating the microbiome to improve life expectancy of PDAC patients.

### SUMMARY

As such, provided herein are studies of the tumor microbiota of independent cohorts of PDAC patients at two geographically disparate tertiary care institutions (studying long-term versus short-term survivors) that demonstrate the host-related influences on long-term survival. Furthermore, methods are provided herein for human fecal microbial transplants from survivors of PDAC and healthy controls to patients with PDAC to modulate the immune system and affect tumor growth.

In one embodiment, provided herein are methods of classifying a patient having pancreatic ductal adenocarcinoma as being either a short-term survivor or a long-term survivor, the method comprising: (a) obtaining a sample of the patient's tumor; (b) detecting the presence of at least three bacterial species in the sample; and (c) classifying the patient having pancreatic ductal adenocarcinoma as being either a short-term survivor or a long-term survivor based on the bacterial species detected.

In some aspects, if the bacterial species detected belong to the Alphaprotebacteria, Sphingobacteria, and/or Flavobacteria class, then the patient is classified as being a long-term survivor. In some aspects, if the bacterial species detected belong to the Clostridia and/or Bacteroides class, then the patient is classified as being a short-term survivor. In some aspects, if the bacterial species detected belong to the Proteobacteria and/or Actinobacteria genus, then the patient is classified as being a long-term survivor. In some aspects, if the bacterial species detected belong to the Pseudoxanthomonas, Streptomyces, Bacillus, and/or Saccharopolyspora taxon, then the patient is classified as being a long-term survivor. In certain aspects, the Bacillus species is Bacillus clausii. In some aspects, step (c) further comprises determining an alpha diversity level based on the bacterial species detected. In some aspects, if the alpha diversity level is higher than a reference level, then the patient is classified as being a long-term survivor. In some aspects, the reference level is an alpha diversity level in a healthy pancreas.

In one embodiment, provided herein are methods of classifying a patient having pancreatic ductal adenocarcinoma as being either a short-term survivor or a long-term survivor, the method comprising: (a) obtaining a sample of the patient's tumor; (b) detecting a level of of CD3+ T cells, CD8+ T cells, and/or Granzyme B+ cells in the sample; and (c) classifying the patient having pancreatic ductal adenocarcinoma as being either a short-term survivor or a long-term survivor based on the level of CD3+ T cells, CD8+ T cells, and/or Granzyme B+ cells detected. In some aspects, if the level of CD3+ T cells, CD8+ T cells, and/or Granzyme B+ cells is higher than a reference level, then the patient is classified as being a long-term survivor. In some aspects, the reference level is a level of CD3+ T cells, CD8+ T cells, and/or Granzyme B+ cells in a healthy pancreas.

In some aspects, the sample is a formalin-fixed, paraffin-embedded sample. In some aspects, the sample is a fresh frozen sample. In some aspects, the methods further comprise reporting the classification of the patient. In some aspects, the reporting comprises preparing a written or electronic report. In some aspects, the methods further comprise providing the report to the patient, a doctor, a hospital, or an insurance company.

In some aspects, if the patient is classified as being a long-term survivor, then the method further comprises performing Whipple surgery on the patient, administering chemotherapy to the patient, and/or administering radiation therapy to the patient. In some aspects, the chemotherapy and/or the radiation therapy can be administered to the patient before and/or after surgery. In some aspects, if the patient is classified as being a short-term survivor, then the method further comprises performing Whipple surgery on the patient, administering chemotherapy to the patient, and/or administering radiation therapy to the patient. In some aspects, the chemotherapy and/or the radiation therapy can be administered to the patient before and/or after surgery.

In some aspects, if the patient is classified as being a short-term survivor, then the method further comprises performing Fecal Microbiota Transplantation (FMT) from a survivor of pancreatic ductal adenocarcinoma to the patient. In certain aspects, the survivor of pancreatic ductal adenocarcinoma is in remission. In certain aspects, the survivor of pancreatic ductal adenocarcinoma has been in remission for at least five years. In certain aspects, the patient is treated with antibiotics prior to the FMT. In certain aspects, the patient is not treated with antibiotics prior to the FMT.

In one embodiment, provided herein are methods of treating a patient having pancreatic ductal adenocarcinoma, the method comprising performing Fecal Microbiota Transplantation (FMT) from a survivor of pancreatic ductal adenocarcinoma to the patient. In some aspects, the patient is treated with antibiotics prior to the FMT. In some aspects, the patient is not treated with antibiotics prior to the FMT. In some aspects, the methods further comprise performing Whipple surgery on the patient. In some aspects, the survivor of pancreatic ductal adenocarcinoma is in remission. In some aspects, the survivor of pancreatic ductal adenocarcinoma has been in remission for at least five years.

In one embodiment, provided herein are methods for inducing intra-tumoral immune cell infiltration in a patient having pancreatic ductal adenocarcinoma, the method comprising performing Fecal Microbiota Transplantation (FMT) from a survivor of pancreatic ductal adenocarcinoma to the patient. In some aspects, the patient is treated with antibiotics prior to the FMT. In some aspects, the survivor of pancreatic ductal adenocarcinoma is in remission. In some aspects, the survivor of pancreatic ductal adenocarcinoma has been in remission for at least five years. In some aspects, the method induces the infiltration of CD8 T cells.

In one embodiment, provided herein are methods for decreasing tumor infiltration by Tregs in a patient having pancreatic ductal adenocarcinoma, the method comprising performing Fecal Microbiota Transplantation (FMT) from a survivor of pancreatic ductal adenocarcinoma or a healthy subject to the patient. In some aspects, the patient is treated with antibiotics prior to the FMT. In some aspects, the patient is not treated with antibiotics prior to the FMT. In some aspects, the survivor of pancreatic ductal adenocarcinoma is in remission. In some aspects, the survivor of pancreatic ductal adenocarcinoma has been in remission for at least five years.

In one embodiment, provided herein are pharmaceutical compositions comprising fecal microbiota obtained from a survivor of pancreatic ductal adenocarcinoma and a pharmaceutically acceptable carrier. In some aspects, the survivor of pancreatic ductal adenocarcinoma is in remission. In some aspects, the survivor of pancreatic ductal adenocarcinoma has been in remission for at least five years.

In one embodiment, provided herein are methods for inducing an immunoactive microenvironment in a pancreatic ductal adenocarcinoma having an immunosuppressive microenvironment, the methods comprising performing Fecal Microbiota Transplantation (FMT) from a survivor of pancreatic ductal adenocarcinoma or a healthy subject to a patient having a pancreatic ductal adenocarcinoma with an immunosuppressive microenvironment. In some aspects, the methods comprise performing FMT from a survivor of pancreatic ductal adenocarcinoma to the patient. In some aspects, the patient is treated with antibiotics prior to the FMT. In some aspects, the patient is not treated with antibiotics prior to the FMT. In some aspects, the survivor of pancreatic ductal adenocarcinoma is in remission. In some aspects, the survivor of pancreatic ductal adenocarcinoma has been in remission for at least five years.

In one embodiment, provided herein are methods of sensitizing a pancreatic ductal adenocarcinoma having an immunosuppressive microenvironment to immune checkpoint inhibitors, the method comprising performing Fecal Microbiota Transplantation (FMT) from a survivor of pancreatic ductal adenocarcinoma or a healthy subject to a patient having a pancreatic ductal adenocarcinoma with an immunosuppressive microenvironment. In some aspects, the methods comprise performing FMT from a survivor of pancreatic ductal adenocarcinoma to the patient. In some aspects, the patient is treated with antibiotics prior to the FMT. In some aspects, the patient is not treated with antibiotics prior to the FMT. In some aspects, the survivor of pancreatic ductal adenocarcinoma is in remission. In some aspects, the survivor of pancreatic ductal adenocarcinoma has been in remission for at least five years. In some aspects, the methods further comprise administering an immune checkpoint inhibitor to the patient.

In one embodiment, provided herein are methods of treating a patient having pancreatic ductal adenocarcinoma, the method comprising (1) performing Fecal Microbiota Transplantation (FMT) from a survivor of pancreatic ductal adenocarcinoma or a healthy subject to the patient and (2) administering an immune checkpoint inhibitor to the patient. In some aspects, the methods comprise performing FMT from a survivor of pancreatic ductal adenocarcinoma to the patient. In some aspects, the patient is treated with antibiotics prior to the FMT. In some aspects, the patient is not treated with antibiotics prior to the FMT. In some aspects, the methods further comprise performing Whipple surgery on the patient. In some aspects, the survivor of pancreatic ductal adenocarcinoma is in remission. In some aspects, the survivor of pancreatic ductal adenocarcinoma has been in remission for at least five years. In some aspects, the immune checkpoint inhibitor targets adenosine A2A receptor (A2AR), B7-H3 (also known as CD276), B and T lymphocyte attenuator (BTLA), cytotoxic T-lymphocyte-associated protein 4 (CTLA-4, also known as CD152), glucocorticoid-induced tumour necrosis factor receptor-related protein (GITR), indoleamine 2,3-dioxygenase (IDO), killer-cell immunoglobulin (KIR), lymphocyte activation gene-3 (LAG3), Mer tyrosine kinase (MerTK), OX40, programmed death 1 (PD-1), programmed death-ligand 1 (PD-L1), T cell immunoreceptor with Ig and ITIM domains (TIGIT), T-cell immunoglobulin domain and mucin domain 3 (TIM-3), or V-domain Ig suppressor of T cell activation (VISTA). In some aspects, the immune checkpoint inhibitor comprises one or more of an anti-PD1 therapy, an anti-PD-L1 therapy, and an anti-CTLA-4 therapy.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-E. Tumor microbial diversity influences the outcome of PDAC patients. (FIG. 1A) Kaplan-Meier plot of MDACC cohort PDAC patients. (FIG. 1B) Alpha diversity box plot (Observed species, Shannon and Simpson reciprocal) in MDACC and JHH cohorts of PDAC patients. (FIG. 1C) Kaplan-Meier plot of MDACC cohort PDAC patients defined by alpha diversity. (FIG. 1D) Principal coordinate analysis (PCoA) using Unweighted-UniFrad of beta diversity. (FIG. 1E) Principal coordinate analysis (PCoA) using Bray-Curtis metric distances of beta diversity.

FIGS. 2A-F. Tumor microbiome communities are significantly different between LTS and STS. (FIG. 2A) Bar plots of the class taxonomic levels in MDA and JHH cohorts of PDAC patients. Relative abundance is plotted for each tumor. (FIG. 2B) Taxonomic Cladogram from LEfSe, depicting taxonomic association from between microbiome communities from LTS and STS PDAC patients. (FIG. 2C) LDA score computed from features differentially abundant between LTS and STS. The criteria for feature selection is Log LDA Score>4. (FIG. 2D) Heatmap of selected most differentially abundant features at the genus level. Highlighting three taxa enriched in LTS. Highlighting three taxa enriched in LTS. (FIG. 2E) Kaplan-Meier estimates for survival probability based on the abundance levels of microbes enriched at Genus level in LTS. Right plot, Saccharopolyspora; middle plot, Pseudoxanthomonas; left plot, Streptomyces (p<0.0001). (FIG. 2F) Plots of differentially abundant genus significantly enriched in both MDA and JHH LTS patients. FDR adjusted p-values.

FIGS. 3A-G. Commensal microbiome from LTS PDAC patients induce a strong immune infiltration and antitumoral immune response. (FIG. 3A) Immunohistochemical CD3, CD8 and Granzyme B, from tumor STS and LTS PDAC patients (representative picture). (FIG. 3B) Quantification of IHC of CD3, CD8 and Granzyme B on STS and LTS PDAC patients. (FIG. 3C) Representative pictures of multiplex immunofluorescence staining (Multiplex IF) with Opal kit. (FIG. 3D) Immunohistochemical CD8 staining from tumor STS and LTS PDAC patients from validation cohorts (JHH) (representative picture). (FIG. 3E) Quantification of the level of CD8 per mm² from the CD8 staining of FIG. 3D. (FIG. 3F) Spearman correlation between CD3+, CD8+ and GzmB+ tissue densities and the overall survival (upper panel) and alpha diversity by Shannon Index (lower panel) of the all PDAC patients. (FIG. 3G) Spearman correlation between CD8+ tissue densities and Saccharopolyspora, Pseudoxanthomonas and Streptomyces (p<0.0001, p=0.006 and p<0.0001, respectively) in PDAC patients.

FIGS. 4A-K. Gut microbiota from PDAC patients can influence tumor microbiota and tumor growth. (FIG. 4A) Taxonomic classification of bacterial 16S sequence by origin present on the unique human stool sample, human adjacent normal to tumor sample, in both stool and normal or absent neither in stool or normal. In the column labeled “Stool”, the top portion represents “Found in Stool and Normal” and the bottom portion represents “Found in Stool”. In the column labeled “Normal Adj”, the top portion represents “Found in Stool and Normal” and the bottom portion represents “Found in Normal”. In the column labeled “PDAC Tumor”, the top portion represents “Not Found in Stool or Normal”, the middle portion represents “Found in Normal”, and the bottom portion represents “Found in Stool”. (FIG. 4B) Experimental design of Fecal Microbiota Transplantation (FMT) from metastatic PDAC donors in C57BL/6 wild-type mouse antibiotic treated (ATBx). (FIG. 4C) Taxonomic classification of bacterial 16S sequence by origin present on the unique human stool PDAC donors, murine stool pre- and post-FMT, or neither in stool donors or murine stool pre-FMT and tumor at the end point. In the column labeled “Donor, Fecal sp.”, the top portion represents “Both Donor or PreTx” and the bottom portion represents “Donor”. In the column labeled “Pre FMT, Fecal sp.”, the top portion represents “Both Donor or PreTx” and the bottom portion represents “PreTx”. In the column labeled “Post FMT, Fecal sp.”, the top portion represents “Neither Donor or PreTx”, the second from the top portion represents “Both Donor or PreTx”, the second from the bottom portion represents “PreTx”, and the bottom portion represents “Donor”. In the column labeled “Post FMT, Tumor”, the top portion represents “Neither Donor or PreTx”, the middle portion represents “PreTx”, and the bottom portion represents “Donor”. In the column labeled “Not FMT, Tumor”, the top portion represents “Neither Donor or PreTx” and the bottom portion represents “PreTx”. (FIG. 4D) Principal coordinate analysis (PCoA) using Unweighted-UniFrad of beta diversity, showing closeness between mice that received FMT from PDAC and distance from those that not received FMT. (FIG. 4E) Experimental design of Fecal Microbiota Transplantation (FMT) from metastatic PDAC, PDAC survivors (PDAC-SV) and Healthy Control (HC) donors in C57BL/6 wild-type mouse antibiotic treated (ATBx). (FIG. 4F) Tumor volume from mice orthotopically implanted with KPC pancreatic cancer cell lines and transplanted with stool from PDAC, PDAC survivors (PDAC-SV) and Healthy Control (HC) donors. (FIG. 4G) Magnetic resonance imaging (MRI) of the mice body orthotopically implanted with KPC pancreatic cancer cell lines and transplanted with stool from PDAC, PDAC survivors (PDAC-SV) and Healthy Control (HC) donors (representative images). (FIG. 4H) Flow cytometry analysis of CD45+CD8+, CD45+CD8+ IFN-γ+, Treg (CD45+CD4+FOXP3+) and MDSC (CD45+CD11b+Ly-6G/Ly-6C+) cells from mice orthotopically implanted with KPC pancreatic cancer cell lines and transplanted with stool from PDAC, PDAC survivors (PDAC-SV) and Healthy Control (HC) donors. (FIG. 4I) Serum level of IL-2 and IFN-γ in mice orthotopically implanted with KPC pancreatic cancer cell lines and receiving stool from PDAC, PDAC survivors (PDAC-SV) and Healthy Control (HC) donors. (FIG. 4J) Experimental design of Fecal Microbiota Transplantation (FMT) from metastatic PDAC survivors (PDAC-SV) donors in C57BL/6 wild-type mouse treated CD8 neutralizing antibodies. (FIG. 4K) Tumor volume from mice orthotopically implanted with KPC pancreatic cancer cell lines and transplanted with stool from PDAC survivors (PDAC-SV) donors in C57BL/6 wild-type mouse treated CD8 neutralizing antibodies.

FIGS. 5A-C. Alpha diversity box plot (Observed species and Shannon) in MDACC cohorts PDAC patients, correlates with (FIG. 5A) Body Mass Index (BMI), (FIG. 5B) sex, and (FIG. 5C) smoking status.

FIGS. 6A-C. Alpha diversity box plot (Observed species and Shannon) in MDACC cohorts PDAC patients, correlates with; (FIG. 6A), neoadjuvant therapies, (FIG. 6B), adjuvants therapies and (FIG. 6C), antibiotics use.

FIGS. 7A-C. Bar plots at the Order taxonomic levels composition in MDACC cohort of PDAC patients, correlating with: (FIG. 7A), Neoadjuvant therapies, (FIG. 7B), Adjuvants therapies and (FIG. 7C), Antibiotics usage prior to surgery (+3 days).

FIG. 8. Sample-wise microbiome ecological distances calculated from a phylogenetic sequencing experiment using Bray-Curtis metric distances.

FIGS. 9A-B. Bar plots at the Phylum (FIG. 9A) and Class (FIG. 9B) taxonomic level composition in MDACC and JHH cohort of PDAC patients. Relative abundance is plotted for each tumor.

FIGS. 10A-B. Bar plots at the Order (FIG. 10A) and Family (FIG. 10B) taxonomic level composition in MDACC and JHH cohort of PDAC patients. Relative abundance is plotted for each tumor.

FIG. 11. Bar plots at the Genus taxonomic level composition in MDACC and JHH cohort of PDAC patients. Relative abundance is plotted for each tumor.

FIGS. 12A-C. (FIG. 12A) Schematic representation of bacterial validation experiments in PDAC frozen tissue. Fluorescence in situ hybridization (FISH) in a human FFPE PDAC samples to detect bacterial 16S rRNA sequences. (FIG. 12B) Cell nuclei were stained with 4′,6-diamidino-2-phenylindole (DAPI). (FIG. 12C) Lipopolysaccharide (LPS) staining in FFPE PDAC samples by immunohistochemistry using an antibacterial-LPS antibody.

FIGS. 13A-E. (FIG. 13A) 16S rDNA PCR was executed using the primers 515F-806R target the V4 region of the 16S rRNA, show the presence of bacterial DNA in the 9 PDAC frozen samples. (FIG. 13B) PCR of Saccharopolyspora genus on PDAC frozen samples. (FIG. 13C) Bar plot of taxonomic composition between FFPE and frozen PDAC samples at the genus level. (FIG. 13D) Culture based assay using frozen PDAC samples. (FIG. 13E) 16S rDNA PCR from colonies selected on agar plate.

FIGS. 14A-B. Bacterial 16S sequence from matched normal adjacent tissue and PDAC samples were classified by taxonomy at Class (FIG. 14A) and Genus (FIG. 14B) levels.

FIGS. 15A-B. (FIG. 15A) Immunohistochemistry (IHC)-based staining of CD66b, FOXP3 and CD68 from tumor STS and LTS PDAC in MDA Cohort (representative pictures). (FIG. 15B) Quantification of IHC staining from FIG. 16A.

FIG. 16. Correlation between Overall survival and Alpha diversity by Shannon Index (top panels) and observed species (bottom panels).

FIGS. 17A-C. (FIG. 17A) Heat map of PICRUSt analysis which identified 26 core functional modules present across all PDAC samples with a coverage of >90% and p<0.05. (FIG. 17B) LDA score computed from enrichment metabolic pathways between LTS and STS. (FIG. 17C) Kaplan-Meier estimates for survival probability based on the top two enrich metabolic pathways in LTS. Upper plot, Xenobiotics Biodegradation and Metabolism (p<0.00001) and lower plot, Lipids Metabolism (p<0.00001).

FIGS. 18A-H. (FIG. 18A) Stacked Bar plots showing taxonomic composition (Order level) on tumors from mice who did not received FMT (no FMT) vs those who received PDAC FMT. (FIG. 18B) Experimental design scheme of the FMT experiment. (FIG. 18C) Beta diversity through LDA comparing gut Microbiome from: HC, PDAC and PDAC SV. (FIGS. 18D-H) Beta-diversity through PCA by Soransen method comparing tumor microbiome from: No FMT vs HC (FIG. 18D), No FMT vs PDAC SV (FIG. 18E), PDAC vs PDAC SV (FIG. 18F), HC vs PDAC SV (FIG. 18G), HC vs PDAC SV (FIG. 18H).

FIGS. 19A-D. (FIG. 19A) Experimental design scheme of the bacterial ablation by antibiotic after FMT. (FIG. 19B) Tumor volume from mice orthotopically implanted with KPC pancreatic cancer cell lines and transplanted with stool from PDAC-SV donors and antibiotic treated after FMT. (FIG. 19C) Taxonomic classification of bacterial 16S sequence by origin present on the unique Donor sample, unique in mice Pre-treatment (PreTx), in both donors and mice or absent neither in donor or PreTx. (FIG. 19D) Principal coordinate analysis (PCoA) using Unweighted-UniFrad of beta diversity, showing closeness between mice that received FMT from PDAC-SV and distance from those that received FMT and were treated with antibiotics post-FMT.

FIG. 20. Serum level of 33 cytokines, chemokines and growth factor in mice who received FMT from PDAC, PDAC-SV and healthy control (HC) patients (who were later challenged with orthotopic PDAC tumors (* p<0.05). For each group of columns, the top represents “HC”, the middle represents “PDAC-SV”, and the bottom represents “PDAC”.

FIGS. 21A-B. (FIG. 21A) Experimental design scheme for CD8 T cell depletion after PDAC SV FMT and tumor challenges. (FIG. 21B) Validation of the CD8 neutralization is shown by flow cytometry reporting that ˜90% depletion was achieved in tumors (right panel) compared to mice who did not receive neutralizing antibodies (left panel).

FIG. 22. AUC curves for the combination of PDAC-microbiome signature using Bacillus genus as a biomarker for long-term survivorship. Left panel is MDACC discovery cohort. Right panel is JHH validation cohort.

FIG. 23. AUC curves for the combination of PDAC-microbiome signature using Bacillus clausii as a biomarker for long-term survivorship. ROC analysis of Taxa relative abundance as predictive of LTS status. The top 3 differential bacteria (genus) identified and Bacillus clausii (one of top species) were tested individually and in aggregate in the MDA discovery cohort (left panel) were then validated in the JHH validation cohort (right panel).

### DETAILED DESCRIPTION

Pancreatic ductal adenocarcinoma (PDAC) is a highly deadly disease, being the fourth leading cause of cancer death in the United States. Most patients diagnosed with resected pancreatic adenocarcinoma (PDAC) survive less than 5-years, but a minor subset of patients survive longer. The factors determining the long-term survival remain elusive. Here, the roles of the tumor microbiota and the immune system in influencing long-term survival were studied. Using 16S rRNA gene sequencing, the tumor microbiome composition in PDAC patients with short and long-term survival (STS, LTS) were analyzed. Higher alpha-diversity was found in the tumor microbiome of LTS patients and an intra-tumoral microbiome signature (Pseudoxanthomonas, Streptomyces, Saccharopolyspora) that consistently distinguishes LTS from STS was analyzed. In addition, greater densities of immune cells were found in PDAC LTS. Remarkably, Fecal Microbiota Transplantation (FMT) from PDAC survivors with no evidence of disease (PDAC SV) into antibiotic-treated mice reduced tumor growth when compared to FMT from PDAC patients or healthy controls donors. Also, PDAC survivor's stools increased intra-tumoral immune cell infiltration, compared with mice who received stools from PDAC patients. Taken together, PDAC microbiome composition influences the host immune response and natural history of the disease.

### I. ASPECTS OF THE PRESENT INVENTION

The microbiota can exert regulatory effects in other sites beyond the gut. The studies provided herein represent the first report to explore the influence of the tumor microbiome on clinical outcomes. A comprehensive analysis of the PDAC intratumoral microbiome was performed in two independent cohorts of long- and short-term survival patients from different institutions. It is important to note that one of these cohorts (JHH) had already been examined for genome wide differences in the mutational landscape that could be contributing to favorable survival and none was identified (Dal Molin et al., 2015). Overall, substantial abundance of microbiome was detected in PDAC tumors from all patients, as previously reported (Geller et al., 2017). PDAC patients with the uncommon phenotype of LTS had significantly higher tumor bacteria diversity than the patients with more typical shorter survival. Further, the LTS and STS cohorts each had a distinctive tumor microbiome signature with specific bacterial genera that were predictive of survival in a multi-variate analysis. Notably, the microbiota reconstitution by FMT with stool from HC, STS, or LTS-NED patients in tumor-bearing mice mirrors the recruitment, or lack thereof, of immune cells to the tumor milieu seen in the respective cohorts, and influences tumor growth, supporting a causal role for the gut microbiome in shaping tumor immune-responses and PDAC progression.

Recent studies have shown that the gut microbiome composition can improve the outcome of cancer immunotherapy (anti-CTLA4 and anti-PD-L1) by influencing the immune system (Gopalakrishnan et al., 2018; Matson et al., 2018; Routy et al., 2018). The present findings suggest that, independent of therapy, the PDAC tumor microbiome diversity and composition can activate specific metabolic pathways and influence immune infiltration, which ultimately influences PDAC survival. Importantly, a signature of 3 tumor bacteria (Saccharopolyspora, Pseudoxanthomonas and Streptomyces) was found to be significantly enriched in LTS patients. Presence of Bacillus clausii, one of the top species enriched in LTS, combined with the three genus signature, was highly predictive of long-term survivorship in the MDA discovery cohort and was validated in the JHH cohort. Tumor microbiome sequencing could be used to stratify patients for adjuvant trials, including microbiome interventions.

Although the greater microbial diversity could have an immunoregulatory impact, its role in the antitumor response is not entirely clear. Components of the Saccharopolyspora family, specifically Saccharopolyspora rectivirgula, have been described as having a role in inflammatory lung diseases, such as hypersensitivity pneumonitis that are associated with IFN-γ overproduction (Kim et al., 2010). The presence of Saccharopolyspora spp. could contribute towards generating a pro-inflammatory microenvironment mediated by cytokines and chemokines that recruit inflammatory cells and IFN-γ secretion. However, its role in PDAC remains to be explored. Future investigations will determine if similar mechanism can be used by the tumor microbiota to modulate the immune system by improving or impairing the immune response against the tumor.

Most of the bacterial communities found in the tumoral milieu are present commonly in the gut microbiome (Human Microbiome Project, 2012; Lloyd-Price et al., 2017), suggesting that bacterial translocation from the gut to the pancreas might be occurring. The studies provided herein show that the gut microbiota has the capacity to colonize pancreatic tumors and that this colonization can modify the overall microbiome of the tumor. Additionally, the present data shows that the modification of the gut/tumor microbiome with human flora from LTS-NED patients is able to induce an antitumor response and activation of the immune system in tumor-bearing mice, which is not observed in FMT from STS patients. Furthermore, immunosuppressive populations were also modulated, with stools from survivors or healthy patients decreasing tumor infiltration by Tregs. This could be a potential mechanism for certain bacteria to promote immune activation. Microbiome-dependent CD8 T cell activation may play a key role. Additionally, the evaluation of cross-reactivity between T cells that recognize tumor neoantigens and microbial antigen (mimicry) present in the tumor may be useful in understanding mechanisms by which bacteria can exert immuno-activating effect but also may be useful in the design on novel therapeutic strategies.

In conclusion, the tumor microbiome diversity has a powerful impact in determining the survival of PDAC patients. The tumor microbiome unique to LTS may contribute towards shaping the favorable tumor microenvironment, characterized by the recruitment and activation of CD8+ T cells to the tumor milieu, and it might also be useful as a predictor of patient outcomes. Besides the microbiome-based prognostic tool, the results of FMT represent an immense therapeutic opportunity to manipulate the microbiome to improve the life expectancy of PDAC patients in whom few therapeutic options exist.

### II. MICROBIOME

The human microbiota consists of trillions of microorganisms including 150-200 prevalent and 1000 less common bacterial species, harboring over 100-fold more genes than those present in the human genome. The microbiota is composed predominantly of bacteria, yet also contains archaea, protozoa, and viruses. The microbiota performs vital functions essential to health maintenance, including food processing, digestion of complex indigestible polysaccharides and synthesis of vitamins, and it secretes bioactive metabolites with diverse functions, ranging from inhibition of pathogens, metabolism of toxic compounds to modulation of host metabolism.

A perturbed microbiota has been implicated in various disorders in humans, from necrotizing enterocolitis in infants, to obesity, diabetes, metabolic syndrome, irritable bowel syndrome, and inflammatory bowel disease in adults. Recent studies of microbiome dysbiosis in human health suggest specific changes in the microbiome in a number of disease states, including cancer. “Microbiome” refers to the collective genomes of a microbiota. Further, studies have suggested the association of a particular microbiome with specific cancers. Thus, a distinct microbiome may contribute to the cause or development of disease. Conversely, the tumor micro-environment may provide a specialized niche in which these viruses and microorganisms may persist. In either case, disease-type specific microbiome signatures may provide biomarkers for early diagnosis, prognosis, and treatment strategies.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of microbes because, although sequences of these genes differ between microbial species, the genes have highly conserved regions for primer binding. This specificity between conserved primer binding regions allows the rRNA genes of many different types of microbes to be amplified with a single set of primers and then to be distinguished by amplified sequences.

### III. DIAGNOSIS, PROGNOSIS, AND TREATMENT OF DISEASES

Detection, isolation, and characterization of the intra-tumoral microbiome, using the methods of the invention, is useful in assessing cancer prognosis and in selecting patients for therapy. This is possible because the intra-tumoral microbiome may be associated and/or correlated with tumor progression and spread, poor response to therapy, relapse of disease, and/or decreased survival over a period of time. Thus, enumeration and characterization of the intra-tumoral microbiome provides methods to stratify patients for baseline characteristics that predict initial risk and subsequent risk based upon response to therapy.

The intra-tumoral microbiome detected according to the methods disclosed herein may be analyzed to diagnose or prognose cancer in the subject. As such, the methods of the present invention may be used, for example, to evaluate cancer patients and those at risk for cancer. In any of the methods of diagnosis or prognosis described herein, either the presence or the absence of one or more indicators of cancer, such as a genomic mutation or intra-tumoral microbiome, or of any other disorder, may be used to generate a diagnosis or prognosis.

In any of the methods provided herein, additional analysis may also be performed to characterize intra-tumoral microbiome to provide additional clinical assessment. For example, image analysis, protein detection techniques, or PCR techniques may be employed, such as multiplexing with primers specific for particular bacterial species. Additionally, DNA or RNA analysis, proteome analysis, or metabolome analysis may be performed as a means of assessing additional information regarding characterization of the patient's intra-tumoral microbiome.

For example, the additional analysis may provide data sufficient to make determinations of responsiveness of a subject to a particular therapeutic regime, or for determining the effectiveness of a candidate agent in the treatment of cancer. Accordingly, the present invention provides a method of determining responsiveness of a subject to a particular therapeutic regime or determining the effectiveness of a candidate agent in the treatment of cancer by detecting intra-tumoral microbiome of the subject as described herein.

For example, the patient may be selected to undergo fecal microbial transplantation. In some embodiments, methods are provided for the treatment or prevention of cancer by the manipulation of the presence, amount, or relative ratio of commensal microflora (e.g., gut microflora). In some embodiments, the presence, amount, or relative ratio of particular bacteria, fungi, and/or archaea within a subject is manipulated. In some embodiments, fecal microbial transplantation utilizes prepared probiotic compositions for administration to a subject. Probiotic compositions comprise one or more beneficial microbes (e.g., bacteria) formulated such that administration of the probiotic (e.g., orally, rectally, by inhalation, etc.) results in population of the subject by the beneficial microbes. The probiotic compositions may be generated from fecal microbiota of a long-term survivor of pancreatic cancer or a healthy subject. In some embodiments, probiotic microbes (e.g., bacteria) are formulated in a pharmaceutically acceptable composition for delivery to a subject. In some embodiments, probiotics are formulated with a pharmaceutically acceptable carrier suitable for a solid or semi-solid formulation. In some embodiments, probiotic microbes are formulated with a pharmaceutically acceptable carrier suitable for a liquid or gel formulation. Probiotic formulations may be formulated for enteral delivery, e.g., oral delivery, or delivery as a suppository, but can also be formulated for parenteral delivery, e.g., vaginal delivery, inhalational delivery (e.g., oral delivery, nasal delivery, and intrapulmonary delivery), and the like.

In some embodiments, donor microflora are obtained by sampling microflora from the desired region of the donor subject body (e.g., colon). In particular embodiments, fecal material (e.g., 100 g-500 g) is obtained from a donor. The material may be administered to a recipient subject with or without subsequent preparation steps (e.g., diluting, mixing, oxygenating, filtering, supplementing (e.g., with prebiotics, with growth media, etc.), testing (e.g., for pathogens or detrimental microbes), etc.). The donor microflora (e.g., fecal material) may be administered without preservation (e.g., administered within 12 hours (e.g., <6 hours, <4 hours, <2 hours, <1 hour, etc.)) or may be preserved (e.g., frozen, freeze dried, etc.), for example, to allow for delay (e.g., 1 day, 2, days, 1 week, 1 month, or more) before delivery to the subject.

In some embodiments, donor microflora are processed to remove one or more components. For example, parasitic of detrimental microbes may be removed or killed. Contaminants within the donor sample may be removed. In some embodiments, donor microflora is enriched for one or more specific microbes (e.g., 2-fold, 3-fold, 4 fold, 10-fold, 20-fold, or more enrichment). In some embodiments, donor microflora is enriched such that at least 1% of the microbes in the population are the desired beneficial microbes (e.g., 1%, 2%, 3%, 4%, 5%, 6%, 7%, 8%, 9%, 10%, 15%, 20%, 25%, 30%, 40%, 50%, 60%, 70%, 80%, 90%, or more). In some embodiments, donor microflora are doped with one or more cultured beneficial microbes.

In particular embodiments, transplanted microflora may be administered to the recipient subject by any suitable delivery mechanism, including but not limited to enema, colonoscope, nasogastric or nasoduodenal tube, lavage or irrigation, or orally (e.g., in the form of a capsule).

The probiotic compositions that find use in embodiments described herein may be formulated in a wide variety of oral administration dosage forms, with one or more pharmaceutically acceptable carriers. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is a mixture with the probiotic microbes. In tablets, the microbes are mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Other forms suitable for oral administration include liquid form preparations such as emulsions, syrups, elixirs, aqueous solutions, aqueous suspensions, or solid form preparations which are intended to be converted shortly before use to liquid form preparations. Aqueous suspensions can be prepared by dispersing the probiotic microbes in water with viscous material, such as natural or synthetic gums, resins, methylcellulose, sodium carboxymethylcellulose, and other well-known suspending agents.

The probiotic compositions (e.g., microbes (e.g., bacteria)) may be formulated for administration as suppositories. A low melting wax, such as a mixture of fatty acid glycerides or cocoa butter is first melted and the probiotic microbes are dispersed homogeneously, for example, by stirring. The molten homogeneous mixture is then poured into conveniently sized molds, allowed to cool, and to solidify.

Rather than pharmaceutical-type formulation, probiotic compositions may be formulated as food additive and/or food product and incorporated into a variety of foods and beverages. Suitable foods and beverages include, but are not limited to, yogurts, ice creams, cheeses, baked products such as bread, biscuits and cakes, dairy and dairy substitute foods, soy-based food products, grain-based food products, starch-based food products, confectionery products, edible oil compositions, spreads, breakfast cereals, infant formulas, juices, power drinks, and the like.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of chemotherapy, immunotherapy, or radiotherapy, performance of surgery, or any combination thereof.

The term “therapeutic benefit” or “therapeutically effective” as used herein refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, a neurodegenerative disease, and/or a genetic disorder).

The terms “contacted” and “exposed,” when applied to a cell, are used herein to describe the process by which a therapeutic agent is delivered to a target cell or are placed in direct juxtaposition with the target cell. To achieve cell killing, for example, one or more agents are delivered to a cell in an amount effective to kill the cell or prevent it from dividing.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Treatment outcomes can be predicted and monitored and/or patients benefiting from such treatments can be identified or selected via the methods described herein.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, the patient's clinical history and response to the agent, and the discretion of the attending physician. The agent is suitably administered to the patient at one time or over a series of treatments.

Therapeutic and prophylactic methods and compositions can be provided in a combined amount effective to achieve the desired effect. A tissue, tumor, or cell can be contacted with one or more compositions or pharmacological formulation(s) comprising one or more of the agents, or by contacting the tissue, tumor, and/or cell with two or more distinct compositions or formulations. Also, it is contemplated that such a combination therapy can be used in conjunction with chemotherapy, radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

A first anti-cancer treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a first anti-cancer therapy is “A” and a second anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311). It is contemplated that one or more anti-cancer therapies may be employed with the antibody therapies described herein.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

Immune checkpoints either turn up a signal (e.g., co-stimulatory molecules) or turn down a signal. Immune checkpoint proteins that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), glucocorticoid-induced tumour necrosis factor receptor-related protein (GITR), indoleamine 2,3-dioxygenase (IDO), killer-cell immunoglobulin (KIR), lymphocyte activation gene-3 (LAG3), Mer tyrosine kinase (MerTK), OX40, programmed death 1 (PD-1), programmed death-ligand 1 (PD-L1), T cell immunoreceptor with Ig and ITIM domains (TIGIT), T-cell immunoglobulin domain and mucin domain 3 (TIM-3), and V-domain Ig suppressor of T cell activation (VISTA). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of Nivolumab (also known as MDX-1106-04, MDX-1106, MK-347, ONO-4538, BMS-936558, and OPDIVO®; described in WO2006/121168), Pembrolizumab (also known as MK-3475, Merck 3475, lambrolizumab, KEYTRUDA®, and SCH-900475; WO2009/114335), Pidilizumab (also known as CT-011, hBAT or hBAT-1; WO2009/101611), Cemiplimab (also known as LIBTAYO®, REGN-2810, REGN2810, SAR-439684, SAR439684), Tislelizumab (also known as BGB-A317, hu317-1/IgG4mt2; U.S. Pat. No. 8,735,553), Spartalizumab (also known as PDR001, PDR-001, NPV-PDR001, NPVPDR001; U.S. Pat. No. 9,683,048), PF-06801591, AK105, BCD-100, BI-754091, HLX10, JS001, LZM009, MEDI 0680, MGA012, Sym021, TSR-042, MGD013, AK104 (bispecific with anti-CTLA4), and XmAb20717 (bispecific with anti-CTLA4).

In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). For example, AMP-224 (also known as B7-DCIg) is a PD-L2-Fc fusion soluble receptor described in WO2010/027827 and WO2011/066342.

In some embodiment, a PD-L1 binding antagonist is an anti-PD-L1 antibody (e.g., a human antibody, a humanized antibody, or a chimeric antibody). In some embodiments, the anti-PD-L1 antibody is selected from the group consisting of Atezolizumab (also known as Tencentriq, MPDL3280A; described in U.S. Pat. No. 8,217,149), Avelumab (also known as BAVENCIO®, MSB-0010718C, MSB0010718C), Durvalumab (also known as IMFINZI®, MEDI-4736, MEDI4736; described in WO2011/066389), FS118, BCD-135, BGB-A333, CBT502 (also known as TQB2450), CK-301, CS1001 (also known as WBP3155), FAZ053, KNO35, MDX-1105, MSB2311, SHR-1316, M7824, LY3415244, CA-170, and CX-072.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, MDX-CTLA4, and YERVOY®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab).

In some embodiment, a CTLA-4 binding antagonist is an anti-CTLA-4 antibody (e.g., a human antibody, a humanized antibody, or a chimeric antibody). In some embodiments, the anti-CTLA-4 antibody is selected from the group consisting of ipilimumab (also known as 10D1, MDX-010, MDX-101, MDX-CTLA4, and YERVOY®; described in WO 01/14424), Tremelimumab (also known as CP-675,206, CP-675, ticilimumab; described in WO 00/37504), BMS-986218, AK104 (bispecific with anti-PD-1), and XmAb20717 (bispecific with anti-PD-1).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP 071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is CD38. The complete protein sequence of human CD38 has Genbank accession number NP 001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Co-stimulatory molecules are ligands that interact with receptors on the surface of the immune cells, e.g., CD28, 4-1BB, OX40 (also known as CD134), ICOS, and GITR. As an example, the complete protein sequence of human OX40 has Genbank accession number NP_003318. In some embodiments, the immunomodulatory agent is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another co-stimulatory molecule that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Yet another co-stimulatory molecule that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immunomodulatory agent is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, HLA-DRB1, HLA-DQA1, HLA-E, killer-cell immunoglobulin (KIR), lymphocyte activation gene-3 (LAG-3, also known as CD223), Mer tyrosine kinase (MerTK), NKG7, programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, immune checkpoint inhibitors targeting the PD-1 axis and/or CTLA-4 have received FDA approval broadly across diverse cancer types.

Other immune inhibitory molecules that can be targeted for immunomodulation include STAT3 and indoleamine 2,3-dioxygenase (IDO). By way of example, the complete protein sequence of human IDO has Genbank accession number NP_002155. In some embodiments, the immunomodulatory agent is a small molecule IDO inhibitor. Exemplary small molecules include BMS-986205, epacadostat (INCB24360), and navoximod (GDC-0919).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### IV. KITS AND DIAGNOSTICS

In various aspects of the invention, a kit is envisioned containing the necessary components to assay a patient's intra-tumoral microbiome. The kit may comprise one or more sealed vials containing any of such components. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass. The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure for intra-tumoral microbiome analysis.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Human samples. First, a discovery cohort of patients with long-term survivors PDAC (>5-years overall survival, median survival 9.5 years, called LTS, n=21) was compared with stage-matched PDAC patients who survive less than 5 years (median survival 1.6 years, called STS, n=22) from University of Texas MD Anderson Cancer Center (MDACC). Detailed clinical and pathologic information on the patients is presented in Table 1. A second validation cohort with long-term survivors of similar characteristics of survival (>5-years overall survival, n=15) compared with stage-matched regular PDAC patients who survive less than 5 years (n=10), from Johns Hopkins Hospital (JHH) (Baltimore, Md.). Archived formalin-fixed paraffin-embedded (FFPE) tumor specimens obtained from patients who underwent surgical resection with curative intent were collected from the University of Texas MD Anderson Cancer Center (Houston, Tex.) and Johns Hopkins Hospital (Baltimore, Md.). The staging of disease was reviewed and updated to comply with the 7th edition of the American Joint Committee on Cancer (AJCC) classification. The study protocol was approved by the Institutional Review Board at MD Anderson Cancer Center.

TABLE 1
Detailed demographic information and clinical characteristics
of all PDAC patients from MDACC cohorts.

| Variable | Level | N | % |
|---|---|---|---|
| Group | LTSM | 21 | 48.8 |
| | STSM | 22 | 51.2 |
| Sex | Female | 20 | 46.5 |
| | Male | 23 | 53.5 |
| Race | White | 39 | 90.6 |
| | Asian | 1 | 2.3 |
| | Black | 1 | 2.3 |
| | Hispanic | 2 | 4.7 |
| BMI Type | Normal | 22 | 51.2 |
| | Obese | 4 | 9.3 |
| | Overweight | 11 | 25.6 |
| | Underweight | 6 | 14 |
| Stage | IB | 3 | 7 |
| | IIA | 16 | 37.2 |
| | IIB | 24 | 55.8 |
| Smoking | Current | 12 | 27.9 |
| | Never | 8 | 18.6 |
| | Past | 23 | 53.5 |
| Drinking | Heavy | 5 | 11.6 |
| | Never | 7 | 16.3 |
| | Occasional | 30 | 69.8 |
| | Past | 1 | 2.3 |
| Neoadjuvant | Yes | 31 | 72.1 |
| | No | 12 | 27.9 |
| Adjuvant | Yes | 25 | 58.1 |
| | No | 18 | 41.9 |
| Recurrence | Yes | 31 | 72.1 |
| | No | 10 | 23.3 |
| | Unknown | 2 | 4.7 |
| Antibiotics Use (pre- | Yes | 17 | 39.5 |
| surgery) | No | 26 | 60.5 |

Stool collection. Stool collection from PDAC patients, PDAC survivors (PDAC-SV), and healthy control (HC) donors were collected on OMNIgene GUT kit (OMR-200) (DNA Genotek, Ottawa, Canada). Fresh stool for fecal microbiota transplantation study from PDAC patients, PDAC survivors (PDAC-SV), and healthy control (HC) donors were collected and frozen at −80° C. prior to FMT.

DNA extraction and bacterial 16S rRNA sequencing. Genomic bacterial DNA extraction methods were optimized to maximize the yield of bacterial DNA while keeping background amplification to a minimum. 16S rRNA gene sequencing methods were adapted from the methods developed for the Earth Microbiome Project (X) and NIH-Human Microbiome Project (Caporaso et al., 2012; Human Microbiome Project, 2012a, b). Briefly, three sections of 10 μm of FFPE of PDAC tissue were aseptically collected and placed in 1.5 mL Eppendorf tubes. Normal pancreatic tissue and paraffin without tissue were used as control. Bacterial genomic DNA was extracted using Qiagen QIAamp DNA FFPE. The 16S rDNA V4 region was amplified by PCR and sequenced in the MiSeq platform (Illumina) using the 2×250 bp paired-end protocol yielding pair-end reads that overlap almost completely. The primers used for amplification contain adapters for MiSeq sequencing and single-index barcodes so that the PCR products may be pooled and sequenced directly (Caporaso et al., 2012), targeting at least 10,000 reads per sample. 16S (variable region 4 [v4]) rRNA gene pipeline data incorporates phylogenetic and alignment-based approaches to maximize data resolution. The read pairs are demultiplexed based on unique molecular barcodes added via PCR during library generation, then merged using USEARCH v7.0.1090 (Edgar, 2010).

Pipeline analysis steps. Raw paired-end 16S rRNA reads (V4 region) were merged into consensus fragments by FLASH (Magoc and Salzberg, 2011) and subsequently filtered for quality (targeted error rate <0.5%) and length (minimum 200 bp) using Trimmomatic (Bolger et al., 2014) and QIIME (Caporaso et al., 2010a; Kuczynski et al., 2011). Spurious hits to the PhiX control genome were identified using BLASTN and removed. Passing sequences were trimmed of primers, evaluated for chimeras with UCLUST (de novo mode) (Edgar et al., 2011), and screened for human-associated contaminant using Bowtie2 (Langmead and Salzberg, 2012). Chloroplast and mitochondrial contaminants were detected and filtered using the RDP classifier (Wang et al., 2007) with a confidence threshold of 50%. High-quality passing 16S rRNA sequences were assigned to a high-resolution taxonomic lineage using Resphera Insight (Daquigan et al., 2017; Drewes et al., 2017) and SILVA Database v128 (Quast et al., 2013). Bacterial contaminant removal was performed using four paraffin-only samples (no tissue). Resulting contaminant-free 16S rRNA profiles were subsampled to 2,000 sequences per sample for downstream comparative analysis. Alpha- and beta-diversity analysis and principal coordinates analysis utilized QIIME and R. Differential abundance analysis of alpha diversity features of interest evaluated differences using the nonparametric difference test. Differential abundance analysis of taxonomic abundances evaluated differences using the negative binomial test (DESeq) (Anders and Huber, 2010). The false discovery rate (FDR) was used to correct for multiple hypothesis testing (Benjamini et al., 2001). Generalized linear modeling adjusting for cohort membership and survival status was performed using R. LEfSe was used for linear discriminant analysis (Segata et al., 2011). High-quality non-contaminant 16S rRNA sequences were analyzed for functional gene content using PICRUSt (Langille et al., 2013), which provides proportional contributions of KEGG categories for each sample (Kanehisa and Goto, 2000). Differentially abundant functional categories (KEGG Level 2, FDR adj.P<0.05 MDA cohort) were utilized for visualization as a heatmap. Relative abundance values were mean centered by functional category and colored according to enrichment or depletion between LTS and STS groups (Magoc and Salzberg, 2011). Statistical annotations were added to denote significant correlations with metadata, enabling quick assessment of many variables. Generated data and figures can be exported as Excel-compatible spreadsheets and publication quality PDFs. These features promote in-depth interrogation of microbial communities by researchers across a wide range of expertise.

Chromogenic immunohistochemistry (IHC) and multiplex immunofluorescence staining (Multiplex IF). 4-μm sections of FFPE tumor tissue were mounted on Superfrost Plus Microscope Slides (FisherScientific) and prepared for IHC and Multiplex IF. The slides were deparaffinized in xylene and rehydrated in graded ethanol. Antigen retrieval was performed in citrate buffer (citrate pH 6.0) using microwave heating (EZ Retriever by BioGenex). Chromogen-based IHC analysis was performed by using antibodies against the following: Polyclonal Rabbit Anti-Human CD3 (T-cell lymphocytes, DAKO, Santa Clara, Calif.), Mouse anti-human CD8 (CD8 T cells, Thermo Fisher Scientific, Waltham, Mass.), mouse anti-human Granzyme B (GzmB, Novocastra, Leica Biosystem), FOXP3 (regulatory T cells, BioLegend, San Diego, Calif.), CD68 (Macrophages, DAKO, Santa Clara, Calif.), and CD66b (Granulocytes/MDSC, BioLegend, San Diego, Calif.). For IHC and Multiplex IF, primary antibody detection was performed using the Opal Polymer HRP Ms+Rb immunohistochemistry detection reagent (PelkinElmer, Boston, Mass.). Signal Stain DAB Substrate KIT (Cell signal, Danvers, Mass.) was used for IHC detection and hematoxylin counterstaining. The densities of cells expressing CD3, CD8 and GzmB were determined by counting cells positive for them in five random square areas (1 mm² each) in the tumor area. The average total number of cells positive for each marker in the five square areas was expressed as density per mm².

Multiplex IF. Staining was performed manually using the same primary antibodies used for IHC analysis against the immune markers: Monoclonal Mouse Anti-Human Cytokeratin AE1/AE3 (Epithelial cell marker, DAKO, Santa Clara), Rabbit Smooth Muscle Actin Polyclonal Antibody (Alpha-smooth muscle isoform of actin, Thermo Fisher Scientific, Waltham, Mass.), Rabbit Anti-Human CD3 (T-cell lymphocytes, DAKO, Santa Clara, Calif.), Mouse anti-human CD8 (CD8 T cells, Thermo Fisher Scientific, Waltham, Mass.), Mouse anti-human Granzyme B (GzmB, Novocastra, Leica Biosystem), Mouse anti-human FOXP3 (regulatory T cells, BioLegend, San Diego, Calif.).

Staining was performed consecutively by using the same steps used in IHC, and the detection for each marker was completed before application of the next antibody. The Opal Polymer HRP Ms+Rb detection reagent (PelkinElmer, Boston, Mass.) was used for the primary antibody detection and Opal 7-Color Manual IHC, with 6 reactive fluorophores (Opal 520, Opal 540, Opal 570, Opal 620, Opal 650, and Opal 690) plus DAPI nuclear counterstain, according to the manufacturer's instructions (catalogue #NEL811001KT, PerkinElmer, Waltham, Mass.). Uniplex IF and Negative control were stained with the same protocols. To the end the slides were imaged using the Vectra 3.0 spectral imaging system (PerkinElmer) according to previously published instructions.

Lipopolysaccharide (LPS) staining and Ribosomal RNA (rRNA) fluorescence in situ hybridization (FISH). Slides were stained for bacteria with the automated slide stainer BOND RXm (Leica) using the Bond polymer refine detection kit, according to manufacturer's instructions. Heat induced epitope retrieval (HIER) at pH6 was done by a 20 min heating step with the epitope retrieval solution 1 (BOND). Gram negative were stained with Lipopolysaccharide Core, mAb WN1 222-5 (1:1000 dilution). FISH was executed using Vysis IntelliFISH Universal FFPE Tissue Pretreatment and Wash Reagents Kit (Abbott Molecular Inc, IL). 5-nm sections of FFPE tumor tissue were hybridized to a probe that recognizes the 16S rRNA genes of all bacteria (green) (Salzman et al., 2010) and counterstained with 4′,6-diamidino-2-phenylindole (DAPI) to visualize nuclei (blue), and tissues were visualized using a Nikon Eclipse Ti microscope.

Flow cytometry. To characterize different subpopulations of immune cells from mice orthotopic PDAC tumors, tumors were removed and digested with Collagenase P to produce single cell suspensions. Single cell suspension was stained with Rat Anti-Mouse CD45 (BD Pharmingen™ PerCP-Cy™5.5), Rat Anti-Mouse CD4 (BD Horizon™ PE-CF594), Rat Anti-Mouse Foxp3 (BD Horizon™ V450), Rat Anti-Mouse Ly-6G (BD Pharmingen™ PE and Ly-6C), Rat Anti-Mouse IFN-γ (BD Pharmingen™ PE), CD8a Monoclonal Antibody (eBioscience™ FITC). Sample acquisition was carried out on LSRFortessa X-20 Analyzer Flow Cytometer (BD Biosciences, Franklin Lakes, N.J.). Analysis was performed with FlowJo version 10 (Tree Star Inc., Ashland, Oreg.).

Tissue culture based and 16S rDNA PCR. Frozen PDAC samples were gently digested with collagenase P in sterile conditions to obtain a single cell suspension. Once the cell fraction was obtained, it was pelleted and the supernatants were plated on Columbia agar and maintained under aerobic and anaerobic conditions overnight at 37° C. The bacterial colonies were selected and DNA was extracted for subsequent PCR amplification of the 16S rDNA using the 515F-806R primers targeting the V4 region of the 16S rRNA. The bacterial 16S rDNA amplified by PCR was subjected to sequencing by the Sanger method. To 16S rDNA PCR in frozen tissue, bacterial DNA was extracted from frozen PDAC tissue samples while maintaining sterility conditions using DNA QIAamp DNA Mini Kit (QIAGEN), and 16S rDNA PCR was executed using the 515F-806R primers targeting the V4 region of the 16S rRNA.

Murine studies. Antibiotic treatments and fecal microbiota transplantation (FMT). All animal experiments were carried out in compliance and approved by the Animal Care and Use Committee at The UT MD Anderson Cancer Center. Female C57BL/6 of 6 were purchased from Taconic Biosciences, USA. Mice were treated for two weeks with an antibiotic solution (ATBx) containing streptomycin (5 mg/mL) and clindamycin (0.1 mg/mL) (Sigma-Aldrich) added to the sterile drinking water of mice. Solutions and bottles were changed 2 times a week. After two weeks of ATBx, treatment was stopped and the mice were recolonized by FMT, receiving stool from patients with PDAC, PDAC-SV, or heathy control donors. Mice received stool 3 times a week by oral gavage using animal feeding needles before undergoing tumor orthotopic implantation and once a week after the tumor implantation until the end point. For CD8+ T cell depletion experiments, the mice were treated for two weeks, 2 times a week by intraperitoneal injection with 150 μg of antibodies against mouse CD8a (Bio X Cell, Lebanon, N.H.). For bacterial ablation experiments, the mice transplanted with stools from PDAC survivors were treated with antibiotics post-FMT in the last two weeks as described earlier.

Orthotopic tumor implantation. C57BL/6 mice were anesthetized with isoflurane. A lateral incision was made on the abdominal wall of each mouse. Each mouse was implanted orthotopically in the pancreas with 2×10⁴ KPC pancreatic adenocarcinoma cells. Tumor growth was monitored by magnetic resonance imaging (MRI) of the mouse body at 4 weeks after the tumor implantation. At the end point, tumors, fecal and blood specimens were harvested and processed for further analysis.

Mouse cytokines, chemokine, and growth factor detection. The quantification of 33 cytokines, chemokines, and growth factor (BCA-1/CXCL13, CTACK/CCL27, ENA-78/CXCL5, Eotaxin/CCL11, Eotaxin-2/CCL24, Fractalkine/CX3CL1, GM-CSF, I-309/CCL1, IFN-γ, IL-1β, IL-2, IL-4, IL-6, IL-10, IL-16, IP-10/CXCL10, I-TAC/CXCL11, KC/CXCL1, MCP-1/CCL2, MCP-2/CCL8*, MCP-3/CCL7, MCP-5/CCL12, MDC/CCL22, MIP-1α/CCL3, MIP-1β/CCL4, MIP-2/CXCL2, MIP-3α/CCL20, MIP-3β/CCL19, RANTES/CCL5, SCYB16/CXCL16, SDF-1α/CXCL12, TARC/CCL17, TECK/CCL25 and TNF-α) were assessed in mouse serum by Bio-Plex Pro™ Mouse Chemokine Assays and Bio-Plex® MAGPIX™ System (Bio-Rad Laboratories, Inc, Hercules, Calif.).

Statistical analyses. The patients' demographic and clinical information was compared using chi-squared test and Fisher's exact test to evaluate the association between two categorical variables. Wilcoxon's rank sum test was used to compare the distributions of continuous variables between two different groups (microbiota composition positive vs. negative). Overall survival (OS) was defined as the time from diagnosis to death from any cause. Patients who did not experience death were censored at the date of last follow-up. Kaplan-Meier curves were estimated for the survival distributions. The Log-rank test was used to test the difference in survival distributions between subgroups. Univariate Cox proportional hazard models were used to determine the effects of microbiota composition on OS (Heller, 2001). Hazard ratios and 95% confidence intervals were provided. All tests were two-sided. P-values less than 0.05 were considered statistically significant. All analyses were conducted using SAS 9.4 (SAS, Cary, N.C.) and S-Plus 8.0 (TIBCO Software Inc., Palo Alto, Calif.) software. Raw 16S rRNA sequences were processed using QIIME (Caporaso et al., 2010b). The minimal sequencing depth was 817, mean was 22178 and maximal was 89566. Sequences were aligned with reference to Silva v128 (Quast et al., 2013). Alpha- and beta-diversity analysis, survival analysis, principal coordinates analysis, ecological network analysis, and Logistic regression combined with LASSO method (https://www.jstor.org/stable/2346178) used R 3.4.3. To LASSO logistic regression, 10-fold cross validations were run with logistic regression for 100 times (starting with different seeds), then all the deviances from 100 validation results were aggregated with respect to each tuning parameter of lambda. The one with minimal average deviance is set as the best lambda value. Then the LASSO logistic regression was fit again with this best lambda value to obtain a stable set of selected features. LEfSe was performed under bioconda environment (available on the world wide web at bioconda.github.io/recipes/lefse/README.html). Linear discriminant analysis (LDA) effect size (LEfSe) (Segata et al., 2011) method was to determine the genomic features most likely to explain differences between biological classes (STS and LTS of MDACC cohorts). Specifically, LEfSe first use the non-parametric factorial Kruskal-Wallis (KW) sum rank test to detect features with significant differential abundance with respect to the Survival term. Biological consistency was subsequently investigated using a set of pairwise tests among subclasses using the (unpaired) Wilcoxon rank-sum test. At last, LEfSe uses LDA to estimate the effect size of each differentially abundant feature. All p-values were adjusted for multiple comparisons with the FDR algorithm (Benjamini et al., 2001). For the discovery, validation and prioritization, the procedure was the following: all genera from the discovery cohort (MDACC) with an FDR adjusted p-value <0.05 between LTS and STS were delimited. Followed by annotation as to whether there was a significant enrichment of each differentially abundant genus in LTS or STS in the discovery cohort. Next, further narrowing the list to those in the validation cohort (JHH) that also have an FDR adjusted p-value <0.05 between LTS and STS was done. Followed by the annotation whether there was a significant enrichment of each differentially abundant genus in LTS or STS in the validation cohort. Subsequently, narrowing of those genera that are concordant for significance and directionality between LTS and STS groups was done. Finally, the prioritization of the list further to those genera that remain significantly associated with LTS/STS status (P<0.01) independent of cohort in a generalized linear model was performed. IHC, Flow cytometry, Mouse Chemokine Assays and tumor size data were analyzed and expressed as the mean±standard deviation using GraphPad Prism 7 (GraphPad Software, Inc., San Diego, Calif.).

### Example 2—Tumor Microbial Diversity is Associated with Better Outcomes in Resected PDAC Patients

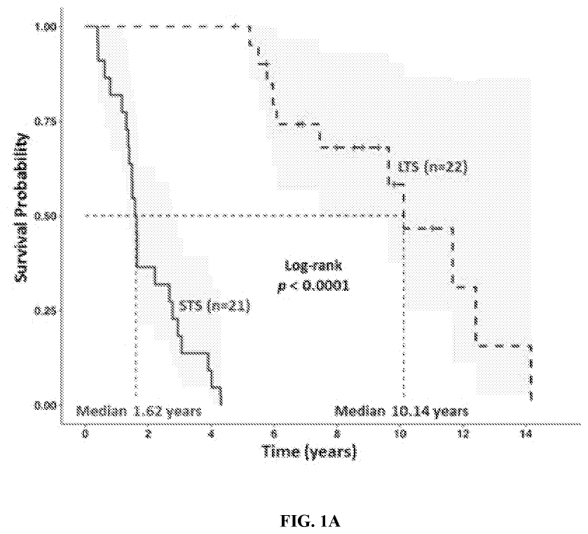

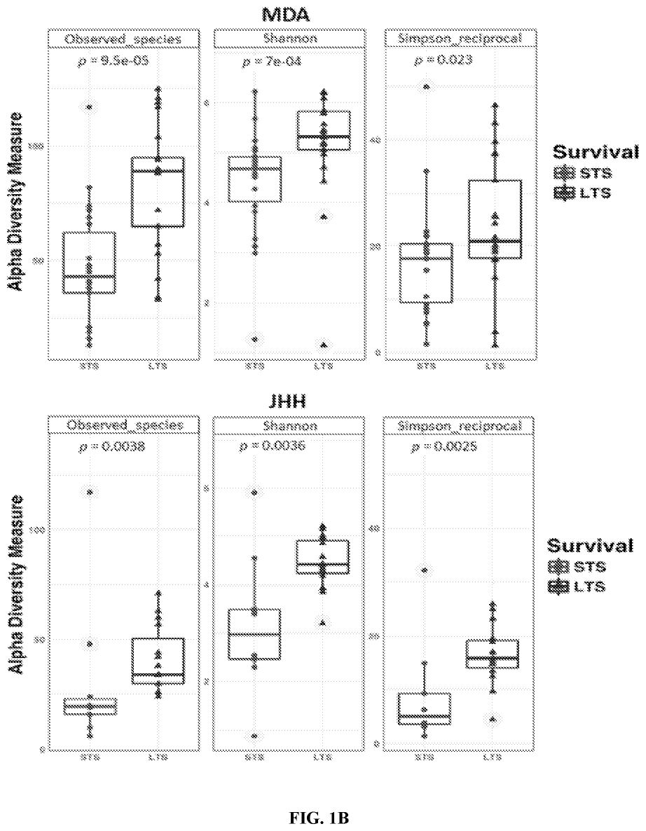

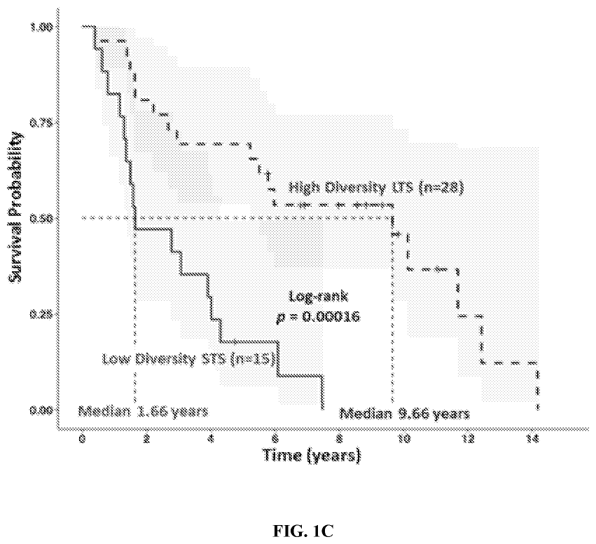

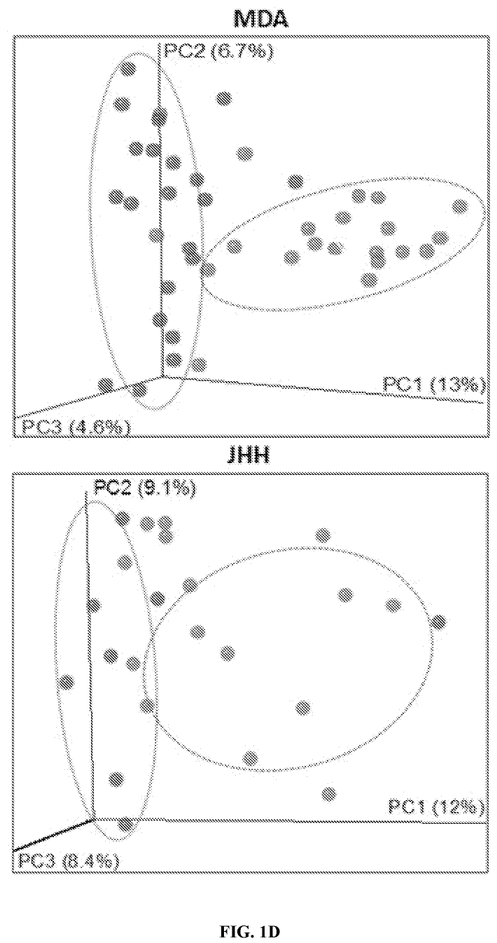

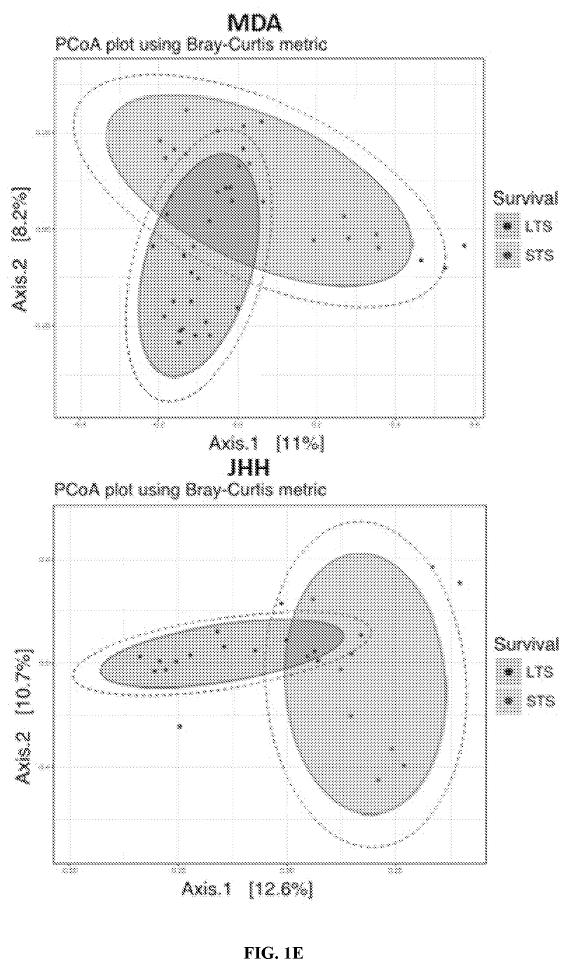

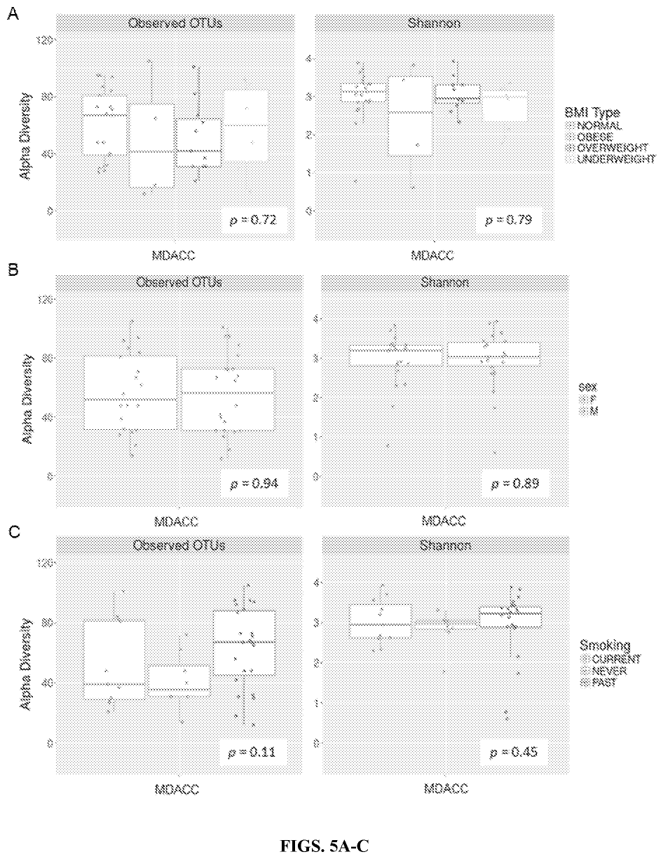

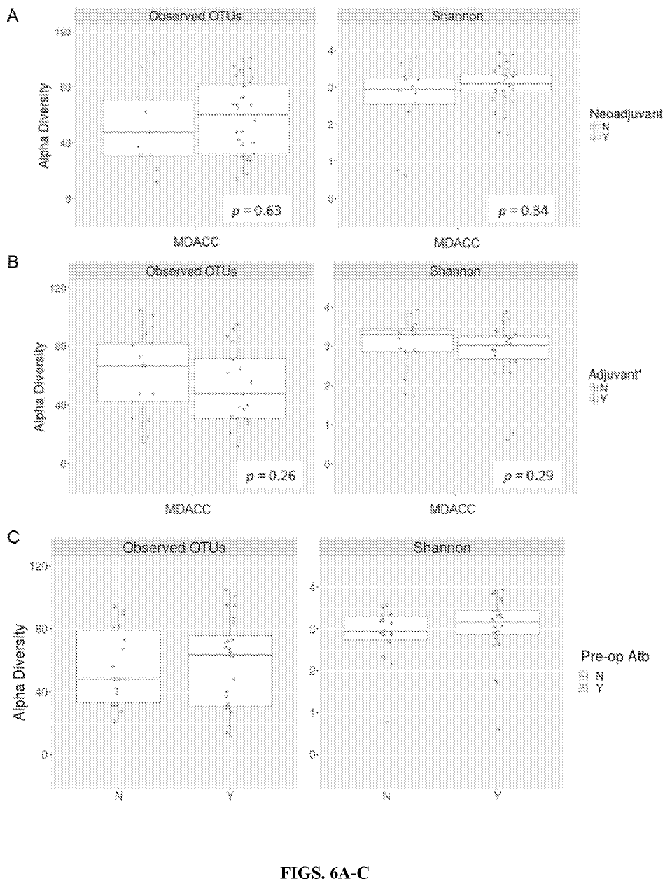

To explore the role of the human tumor microbiome composition in mediating clinical outcomes of PDAC patients, a discovery cohort was used to compare surgically resected patients who survived more than 5 years post-surgery, or long-term survivors (LTS, median survival 10.1 years), to stage-matched short-term survivors who survived less than 5 years post-surgery (STS, median survival 1.6 years) from UT MD Anderson Cancer Center (MDACC) in Houston, Tex. (FIG. 1A and Table 1). Patients in LTS and STS groups were similar with respect to age, gender, stage, and prior therapies, including antibiotic use and neoadjuvant or adjuvant treatments (Table 2). Then, a validation cohort with similar survival characteristics from Johns Hopkins Hospital (JHH) in Baltimore, Md. was used. Bacterial DNA was extracted from 68 surgically resected PDAC tumor (36 LTS and 32 STS) and taxonomic profiling via 16S rRNA gene sequencing was performed. First, the tumor microbial diversity was measured using different methodologies (Observed Taxonomic Units, Shannon and Simpson Indices) and it was found that alpha-diversity of the tumor microbiome, defined as the number of species present within each tumor sample (Kurilshikov et al., 2017), was significantly higher in the LTS patients compared to STS on both the MDACC discovery cohort (p<0.0005, p<0.0005 and p<0.05, for each alpha-diversity index, respectively) and the JHH validation cohort (p<0.005, p <0.005 and p<0.005, for each alpha-diversity index, respectively) (FIG. 1B). Based on these results, the relationship between PDAC tumor microbial diversity and overall survival (OS) in the MDACC cohort was tested by stratifying the patients into two groups based on median diversity obtained by Shannon index. As expected, patients with high alpha diversity had significantly prolonged overall survival (median survival: 9.66 years) compared to those with low alpha diversity (median survival: 1.66 years), as determined using univariate Cox proportional hazard models (FIG. 1C and Table 3, p<0.0005). The relationship between tumoral microbial diversity and survival actually allowed for the stratification and redistribution of PDAC patients according to alpha diversity value (high or low) (FIG. 1C). Importantly, potential contributors to microbial diversity were assessed, including clinico-pathological features, body mass index, sex, smoking, neoadjuvant/adjuvant therapies as well as antibiotics use, and no significant associations were found (FIGS. 5A-6C). Recent studies have proposed that a high microbial diversity in the gut microbiome is associated with favorable outcomes to treatment (Gopalakrishnan et al., 2018). On the contrary, an imbalance in the gut microbiome, or dysbiosis, is associated with poor responses to these therapies, and associated with chronic diseases and cancer development (Ferretti et al., 2017; Human Microbiome Project, 2012; Kundu et al., 2017; Lloyd-Price et al., 2017; Shoemark and Allen, 2015). Thus, tumor alpha diversity could serve as a predictor of survival outcome in resected PDAC patients, suggesting the potential relevance of the microbiome composition in mediating pancreatic cancer progression.

To extend the understanding of the role of microbiome diversity and its association with survival, detection of whether phylogenetic relationships exist between the bacterial communities enriched in the PDAC milieu of STS and LTS was sought. Beta-diversity was used to generate a principal coordinate analysis (PCoA) using Unweighted-UniFrac distances (Lozupone et al., 2011) and using Bray-Curtis metric distances (McMurdie and Holmes, 2013). A clear clustering between operational taxonomic units (OTUs) from LTS and STS was revealed by both methods in both independent cohorts (FIGS. 1D-1E & 8), suggesting that the tumor microbial communities exhibit phylogenetic closeness within each group (p<0.05).

TABLE 2
Characteristics of patients involved in this study.

| Patient Characteristics | | STS (n = 22) | LTS (n = 21) | P value |
|---|---|---|---|---|
| Overall Survival (yrs) | | 1.62 | 10.14 | <0.0001 |
| Surgery Date | | 1999-2014 | 2000-2010 | |
| Gender | Female | 9 | 11 | 0.54 |
| | Male | 13 | 10 | |
| Age (yrs) | Median | 62.05 | 62.71 | 0.69 |
| | Range | 46-74 | 44-73 | |
| Race | Caucasian | 19 | 20 | 0.48 |
| | Asian | 0 | 1 | |
| | African American | 1 | 0 | |
| | Hispanic | 2 | 0 | |
| Stage | IB | 2 | 1 | 0.56 |
| | IIA | 7 | 10 | |
| | IIB | 13 | 10 | |
| Neoadjuvant Therapy | Yes | 14 | 17 | 0.31 |
| | No | 8 | 4 | |
| Adjuvant Therapy | Yes | 14 | 11 | 0.45 |
| | No | 8 | 10 | |
| Antibiotics Use (pre-surgery) | Yes | 14 | 11 | 0.45 |
| | No | 8 | 10 | |
| Biliary Obstruction | Yes | 12 | 10 | 0.64 |
| | No | 10 | 11 | |

TABLE 3
Association between survival and tumor microbiome
diversity (Observed and Shannon index).

| Alpha | Variable | | | | | | |
|---|---|---|---|---|---|---|---|
| Diversity | HazardRatio | HRLowerCL | HRUpperCL | pValue | Event | Censored | Total |
| Observed | 0.9819 | 0.9709 | 0.9931 | 0.00016 | 32 | 10 | 42 |
| Species | | | | | | | |
| Shannon | 0.6805 | 0.5248 | 0.8823 | 0.0037 | 32 | 10 | 42 |
| Index | | | | | | | |

### Example 3—Tumor Microbiome Communities are Significantly Different Between LTS and STS

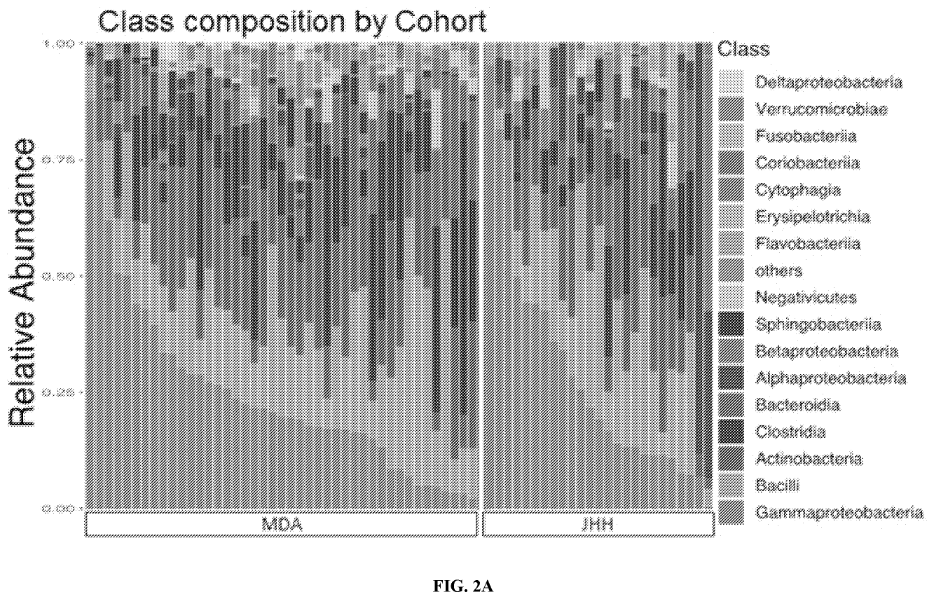

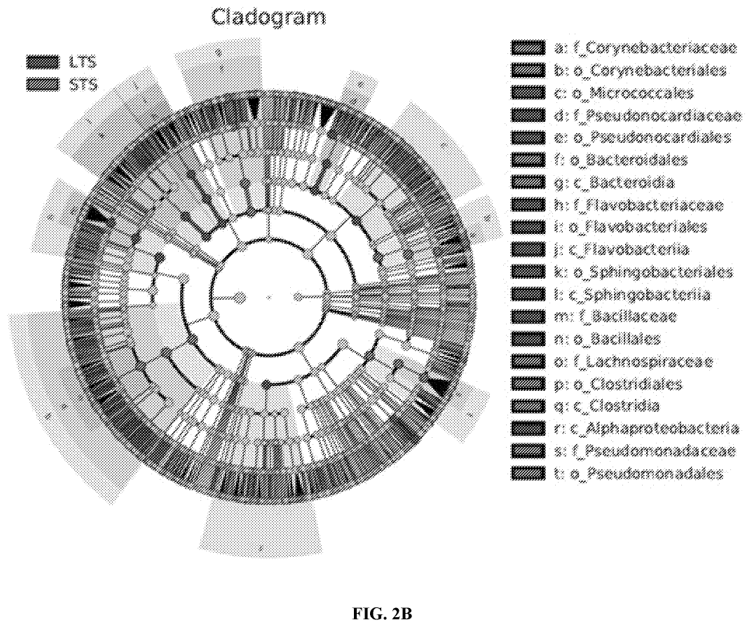

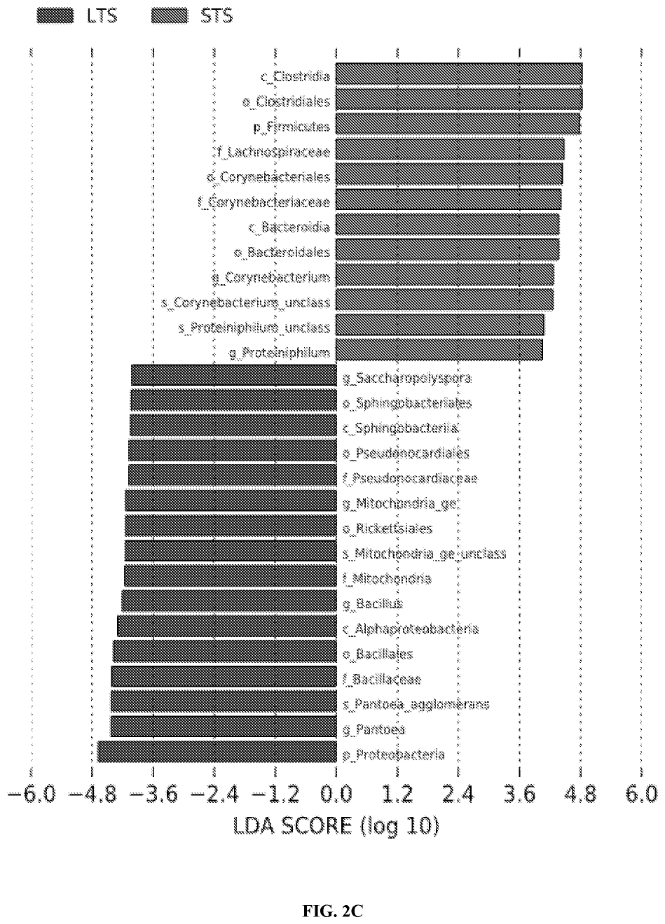

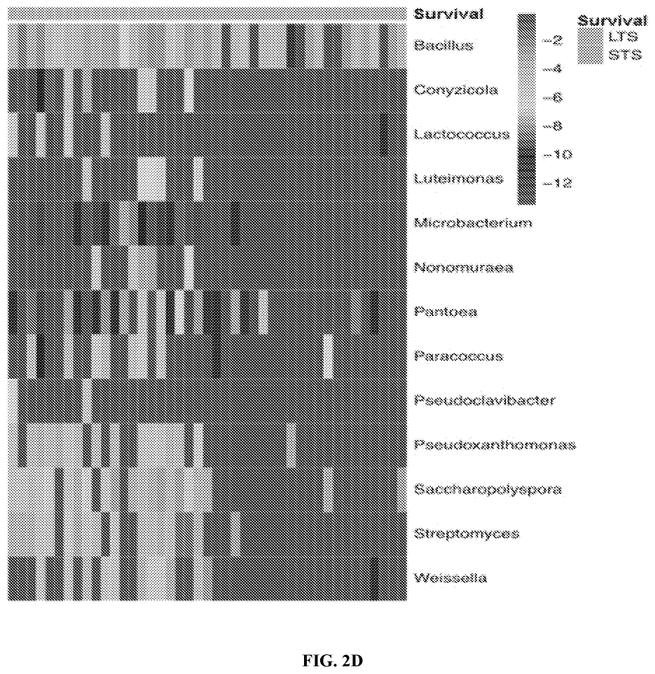

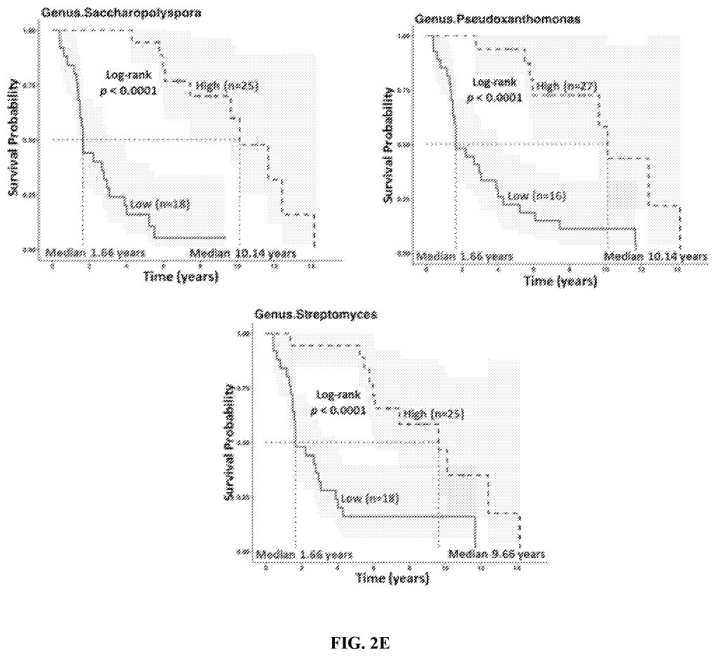

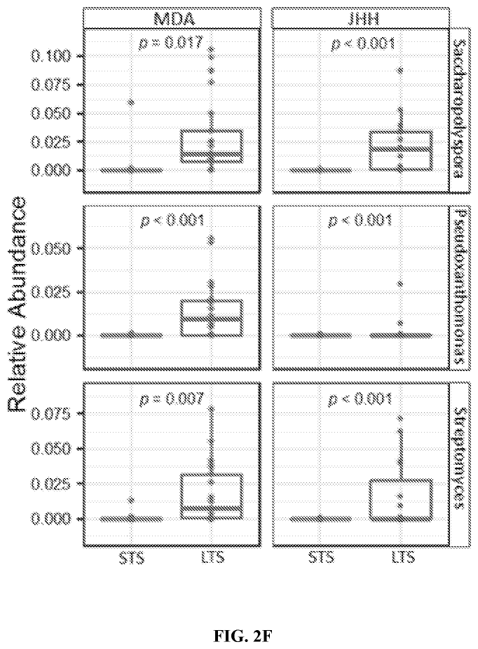

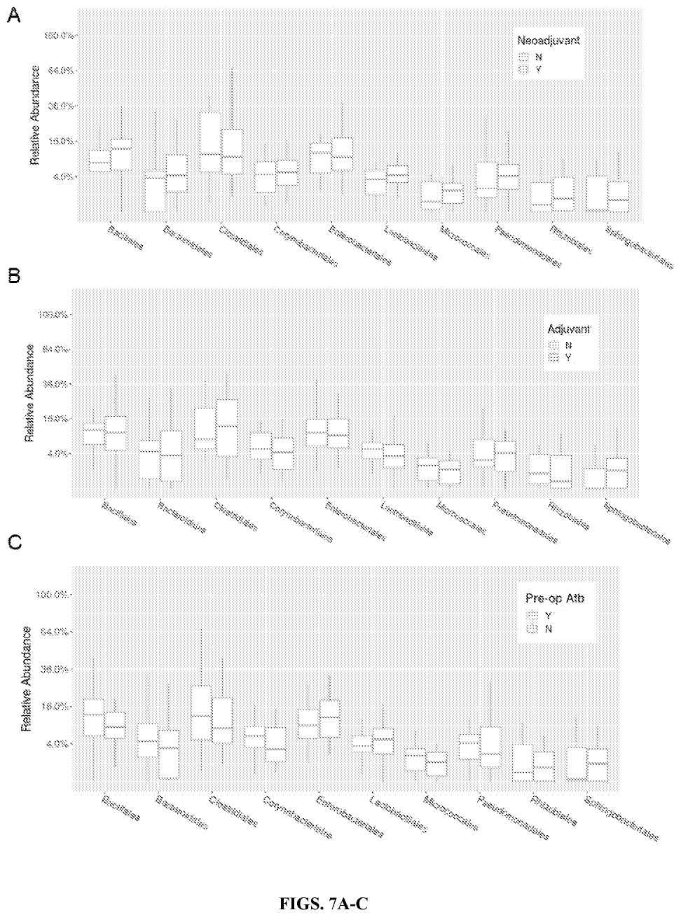

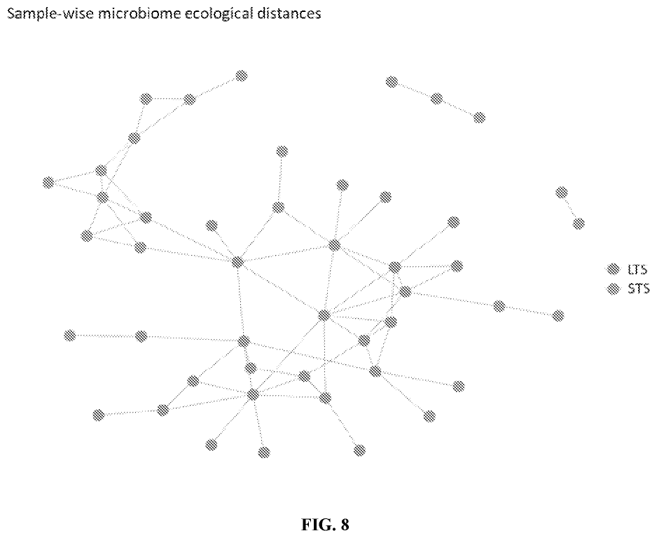

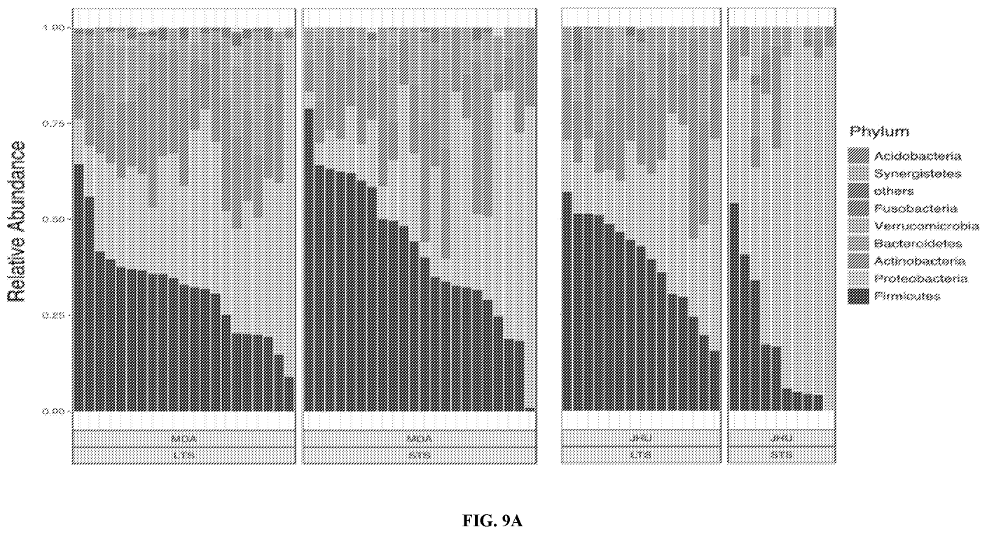

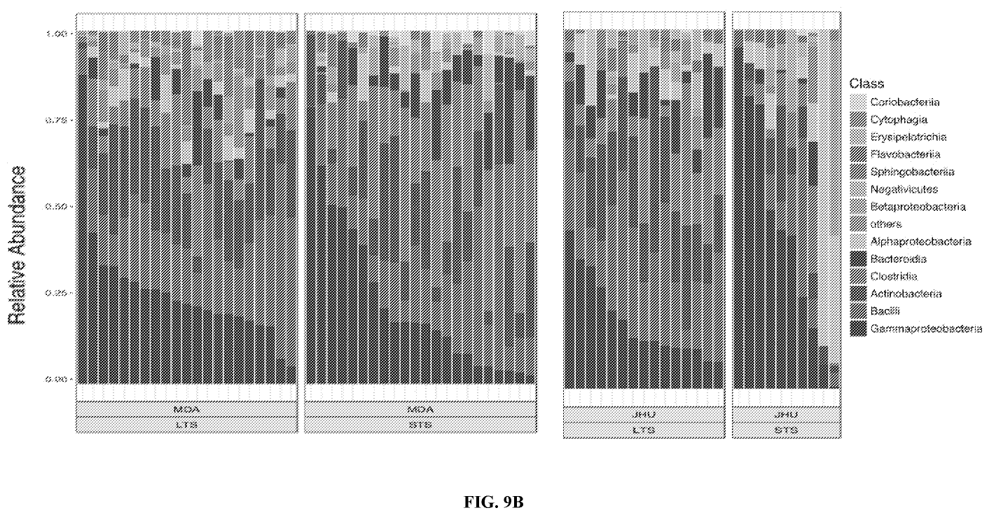

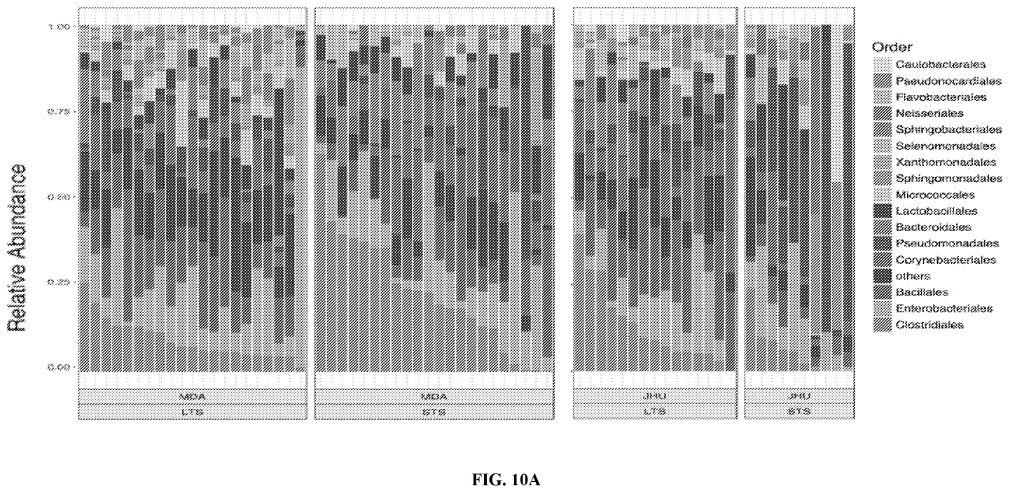

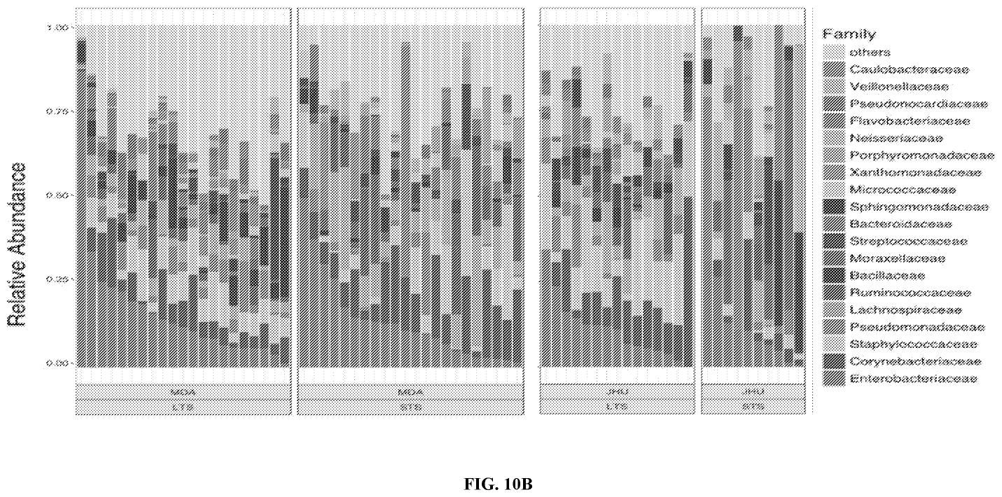

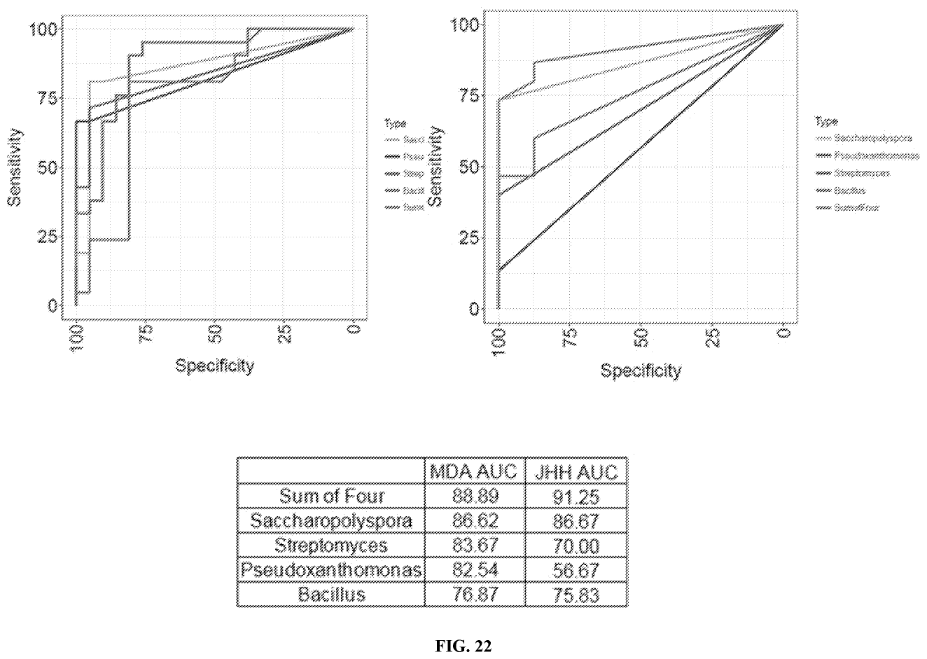

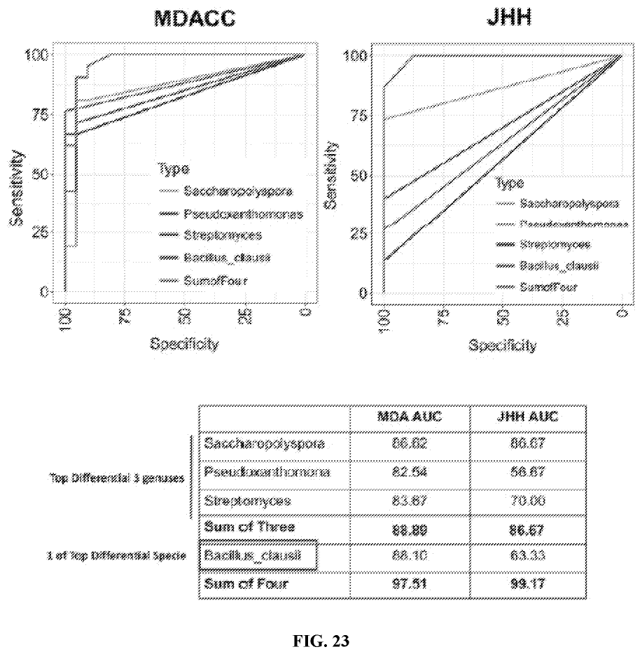

Considering the relationship between PDAC intra-tumoral bacterial diversity and overall survival, whether there were differences in the tumor microbiome composition between PDAC LTS and STS was next determined. To this end, the general landscape of the tumor microbiome on all patients from both cohorts was assessed, which revealed presence of similar communities (FIG. 2A). Then, enrichment of OTUs in LTS versus STS was compared, which revealed enrichment for specific bacterial communities in each group at the various taxonomic levels (FIGS. 9-11). When the influence of neoadjuvant and adjuvant therapies and antibiotics use in the MDACC cohort were evaluated, no significant differences were detected in the taxonomic composition at the order level (FIGS. 7A-C). To further investigate these findings, the discovery cohort was used to conduct high dimensional class comparisons using linear discriminant analysis of effect size (LEfSe) (Segata et al., 2011), which detected marked differences in the predominance of bacterial communities between LTS and STS (FIGS. 2B-2C). The LTS tumors exhibited a predominance of Alphaprotebacteria, Sphingobacteria, and Flavobacteria at the class level. In contrast, the PDAC STS cases were dominated by Clostridia and Bacteroidea at the class level (FIGS. 2B, 2C). Then, it was determined whether the tumor microbiome could be segregated by comparison heatmap based in the OTU abundance at the genus level using patients' survival as a variable (FIG. 2D). The genus features were selected using logistic regression combined with LASSO methods. The taxonomic community's differential segregation was visualized according to the survival of the PDAC patients. The LTS patients showed an enrichment on Proteobacteria (Pseudoxanthomonas) and Actinobacteria (Saccharopolyspora and Streptomyces), while no predominant genus was detected in the STS tumors. Then, patients were stratified into high versus low categories based on the median relative abundance of these three taxa (Pseudoxanthomonas, Saccharopolyspora, and Streptomyces). Significantly better outcomes were predicted for PDAC patients with a higher abundance of Saccharopolyspora (HR=13.47, 95% CI 4.672-38.83), Pseudoxanthomonas (HR=5.885, 95% CI 2.37-14.61), and Streptomyces (HR=4.572, 95% CI 2.033-10.28) (FIG. 2E). Considering that all of the above comparisons were done in the discovery cohort, the relative abundance of the top 3 hits was validated in the JHH cohort and it was found that Pseudoxanthomona, Saccharopolsypora, and Streptomyces were also significantly more abundant in LTS vs STS from this cohort (FIG. 2F). These three genera with higher abundance in LTS were then used to run area under curve (AUC)-receiver operator characteristic (ROC) analysis. The combination of these Top 3 taxa (Pseudoxanthomonas, Saccharopolyspora, and Streptomyces) resulted in an AUC of 88.89 in the discovery cohort and 86.67 in the validation cohort (FIGS. 22 and 23).

Next, the differences in mean relative abundance of species between LTS and STS were assessed to determine if any could potentially increase predictive value of long-term survivorship and two species were found to be significantly enriched in LTS versus STS in the discovery cohort: Bacillus clausii (1.78% versus 0%, false discovery rate [FDR] adjusted [adj] p value: 0.001) and Saccharopolyspora rectivirgula (1.91% versus 0.26%, FDR adj p value: 0.001). Because Bacillus clausii belongs to a genus different from the three described, whether its addition to the signature to predict LTS was tested. While individual AUC were 88.10 and 63.3 in the discovery cohort and validation cohorts, respectively, when Bacillius clausii was added to the three genus signature, the AUC in the discovery cohort increased to 97.51 and in validation cohort to 99.17 (FIG. 23). The presence and abundance of these three taxa communities Pseudoxanthomonas, Saccharopolyspora, and Streptomyces, added with the presence of Bacillus clausii, could influence and predict long-term survivorship in PDAC patient. The presence of any one of Pseudoxanthomona, Saccharopolsypora, Streptomyces, and Bacillus, as well as the combination of all four, were able to predict long-term survivorship in both the discovery and validation cohorts (FIG. 22). The use of Bacillus clausii increased the power of the biomarkers (FIG. 23).

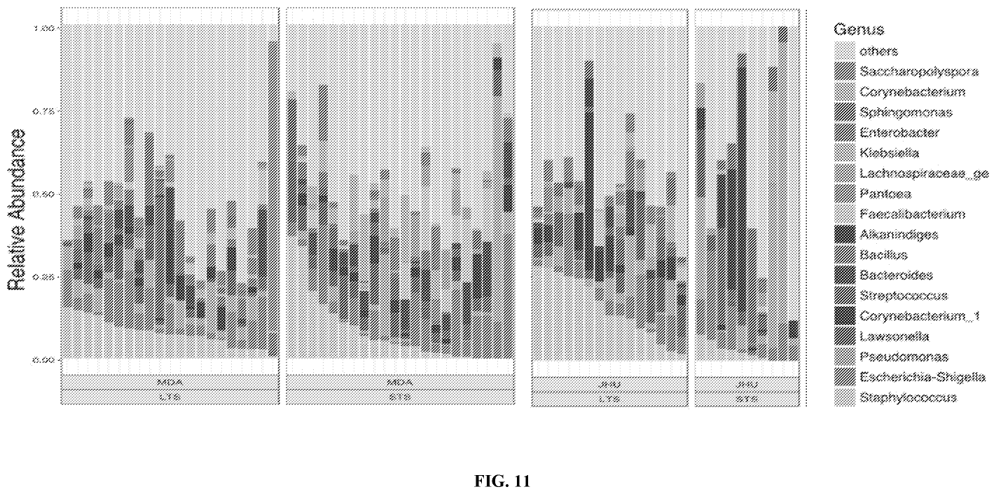

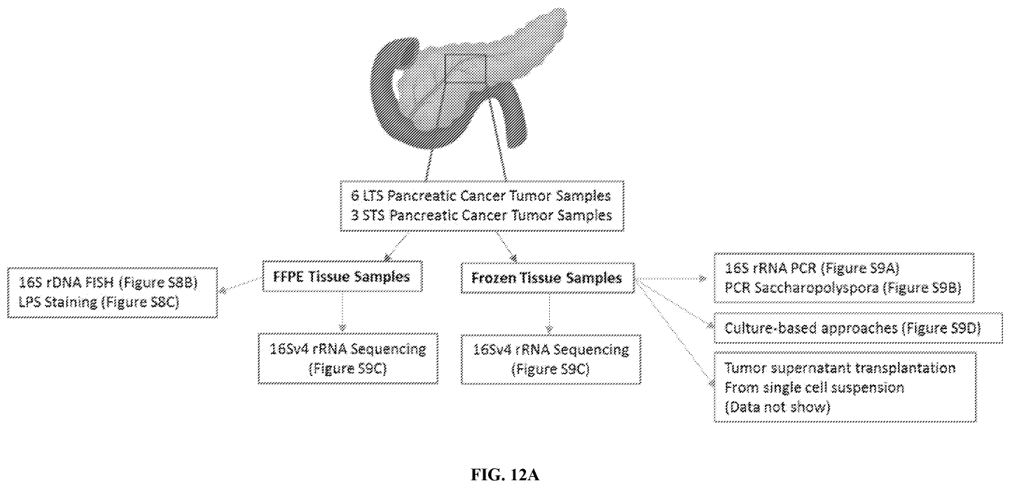

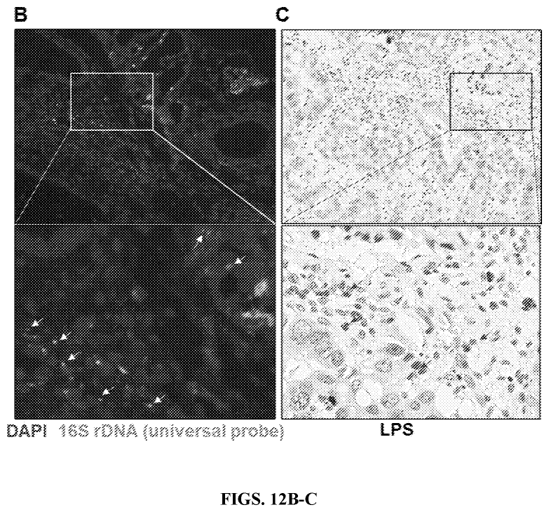

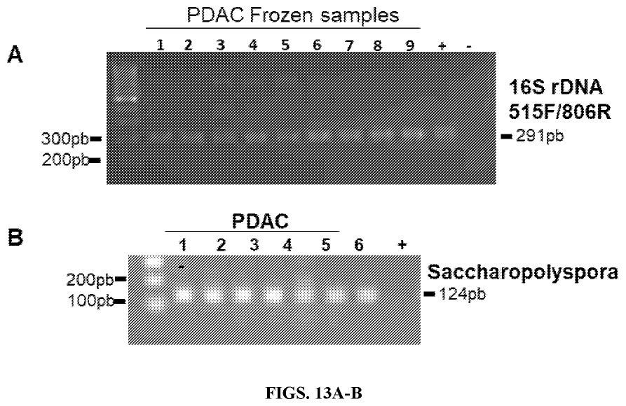

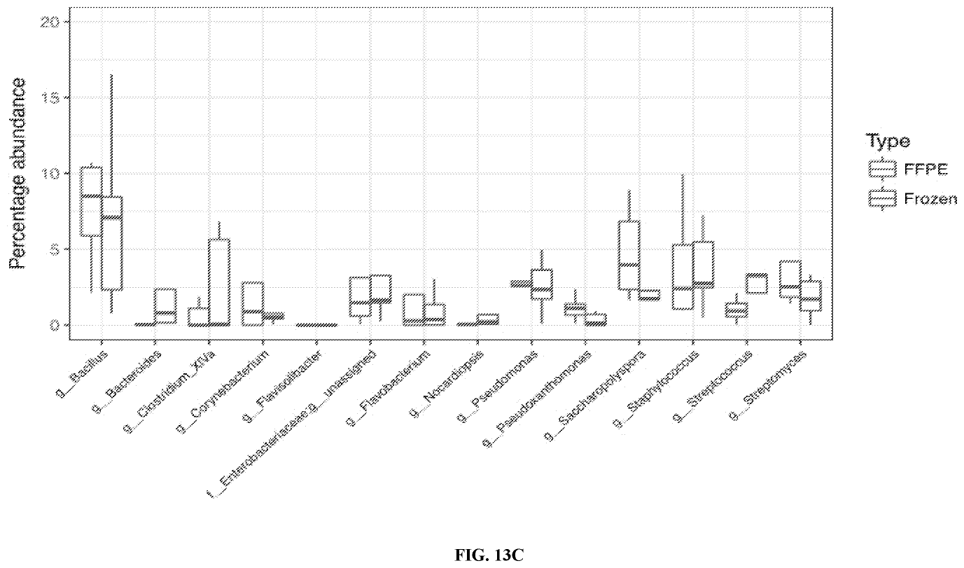

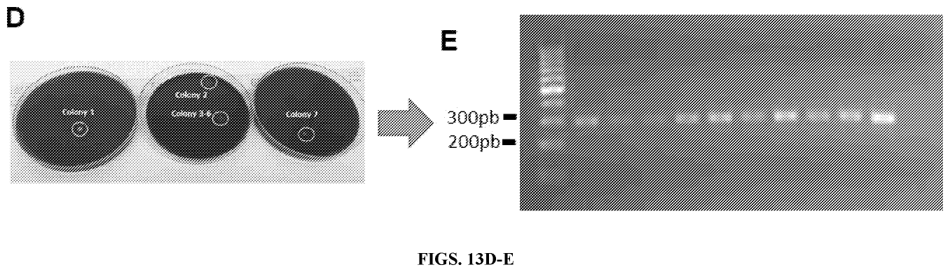

To definitively confirm the presence of intratumoral bacteria in PDAC cases, several additional experiments were conducted (FIG. 12A). First, ribosomal RNA (rRNA) fluorescence in situ hybridization (FISH) was performed in a subset of archival FFPE PDAC samples, using a specific probe that targets bacterial 16S rRNA. The FISH analysis of bacterial 16S rRNA confirmed the presence of bacterial DNA in all PDAC samples analyzed (FIG. 12B). Additionally, in the same set of FFPE samples, the presence of bacterial lipopolysaccharide (LPS) was detected by immunohistochemistry using an antibacterial lipopolysaccharide antibody, as previously performed (Geller et al., 2017). Consistently, this approach confirmed the presence of intratumoral bacteria in all PDAC samples evaluated (FIG. 12C). Furthermore, to verify the presence of bacteria in frozen samples, nine frozen PDAC tissue samples were analyzed that matched FFPE specimens that were previously analyzed. The 16S rDNA PCR demonstrated the presence of bacterial DNA in the nine PDAC frozen samples analyzed (FIG. 13A). Additionally, the presence of Saccharopolyspora genus was assessed using primers designed to amplify several bacterial species from the Saccharopolyspora genus abundantly expressed in LTS and the presence of Saccharopolyspora genus was detected in 6/6 LTS PDAC frozen samples analyzed (FIG. 13B).

Additionally, to confirm whether the overall tumor bacterial composition was similar between frozen and FFPE samples, and to exclude the possibility that most of the bacteria previously found were an artifact of FFPE fixation, 16S rRNA gene sequencing of frozen tissue was performed and compared to the corresponding FFPE samples (FIG. 13C). The data showed similar taxonomic composition between FFPE and frozen PDAC samples, and notably, no statistically significant differences were found between the taxonomic compositions of both types of samples (FIG. 13C and Table 4). Additionally, frozen PDAC samples were used to obtain and isolate bacteria from the tissue (FIG. 13D). These colonies were selected and DNA was extracted for subsequent PCR amplification of the 16S rDNA (FIG. 13E).

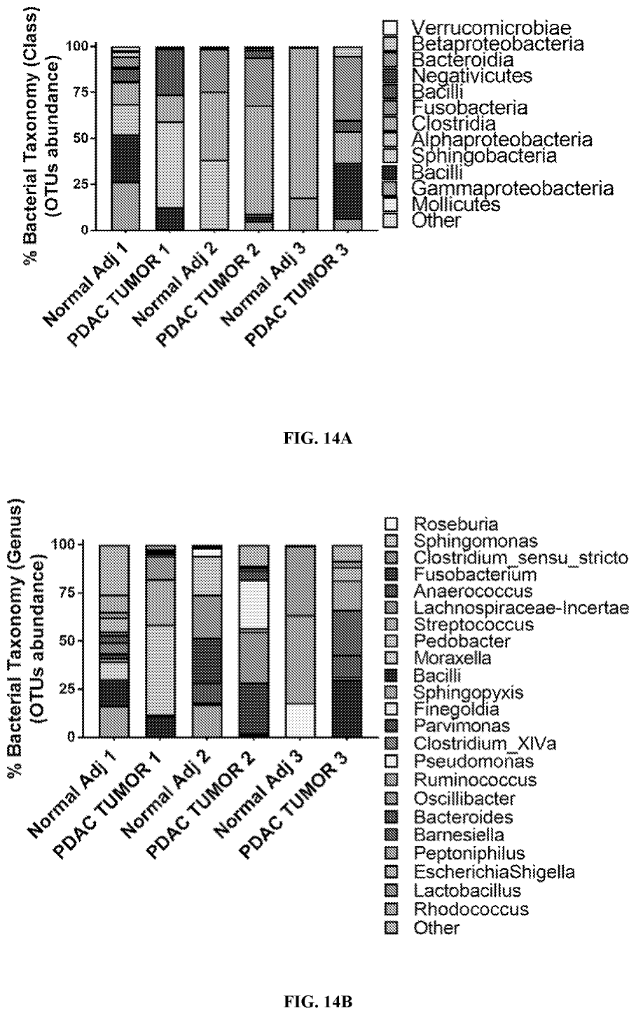

Finally, to understand if the tumor microbiome is unique or similar to its adjacent normal tissue, three matched samples of tumor tissue and its corresponding normal tissue adjacent to the tumor were collected. Bacteria were found in the adjacent normal tissue but their composition was different than in the tumors, suggesting that the tumor microbiome might be unique (Pushalkar et al., 2018) (FIGS. 14A-14B). Overall, these results confirmed the presence of bacteria in the PDAC samples.

TABLE 4
Contaminants List from FFPE samples. Taxa filtered out as contaminants
based on 4 negative controls and additional literature prior to downstream
statistical analysis. All low frequency species/OTUs with fewer than
5 observations across the entire dataset were removed.

Anoxybacillus
Comamonadaceae
EscherichiaShigella
Paenibacillaceae
Geobacillus
Geodermatophilaceae
Hydrogenophilales
Methylobacterium
Oceanospirillales
Paenibacillus
Propionibacterium
Rhizobiales
Thermoactinomyces
Thermoactionomycetaceae
Thermobifida
Thermobispora
Thermophilic
Thermopolyspora
Oxalobacteraceae
Phyllobacteriaceae
Agrobacterium
Averyella_{—}dalhousiensis: Enterobacter_{—}aerogenes:
Enterobacter_{—}asburiae: Enterobacter_{—}cancerogenus:
Enterobacter_{—}cloacae: Enterobacter_{—}hormaechei:
Enterobacter_{—}ludwigii: Erwiniw_{—}aphidicola:
Erwinia_{—}persicina: Erwinia_{—}rhapontici:
Klebsiella_{—}granulomatis: Klebsiella_{—}oxytoca:
Klebsiella_{—}pneumoniea: Klebsilla_{—}singaporensis:
Klebsiella_{—}variicola: Kluyvera_{—}ascorbata:
Kluyvera_{—}cryocrescens: Kluyvera_{—}intermedia:
Lelliottia_{—}amnigena: Pantoea_{—}agglomerans:
Raoultella_{—}terrigena
Corynebacterium_{—}pseudogenitalium:
Corynebacterium_{—}tuberculostearicum
Dietzia_{—}alimentaria
Pseudomonas_{—}antarctica: Pseudomonas_{—}auricularis:
Pseudomonas_{—}azotoformans: Pseudomonas_{—}brenneri:
Pseudomonas_{—}cedrina: Pseudomonas_{—}costantinii:
Pseudomonas_{—}extremaustralis: Pseudomonas_{—}extremorientalis:
Pseudomonas_{—}fluorescens: Pseudomonas_{—}gessardii:
Pseudomonas_{—}libanensis: Pseudomonas_{—}lurida:
Pseudomonas_{—}meridiana: Pseudomonas_{—}poae:
Pseudomonas_{—}putida: Pseudomonas_{—}reactans:
Pseudomonas_{—}salomonii: Pseudomonas_{—}syncyanea:
Pseudomonas_{—}synxantha: Pseudomonas_{—}tolaasii:
Pseudomonas_{—}trivialis: Pseudomonas_{—}veronii
Staphylococcus_{—}aureus: Staphylococcus_{—}capitis:
Staphylococus_{—}caprae: Staphylococcus_{—}devriesei:
Staphylococcus_{—}epidermidis: Staphylococcus_{—}haemolyticus:
Staphylococcus_{—}hominis: Staphylococcus_{—}jettensis:
Staphylococcus_{—}lugdunensis: Staphylococcus_{—}pasteuri:
Staphylococcus_{—}petrasii: Staphylococcus_spC10c:
Staphylococcus_{—}warneri

### Example 4—the Tumor Microbiome Shapes Immune Responses Promoting T Cell Activation

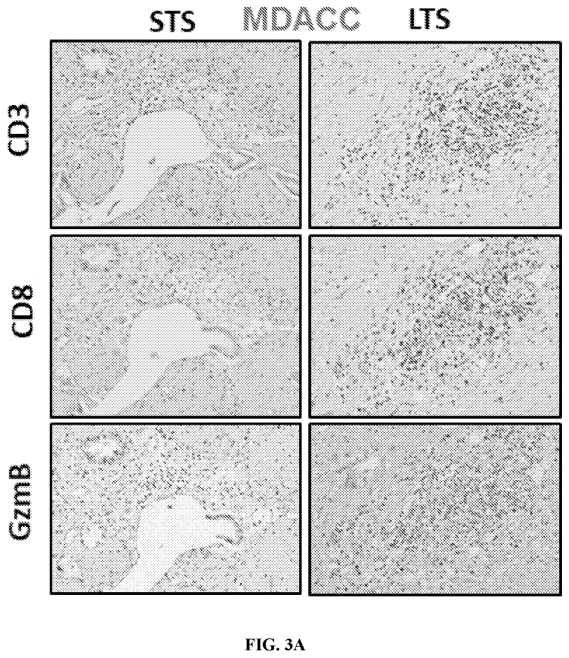

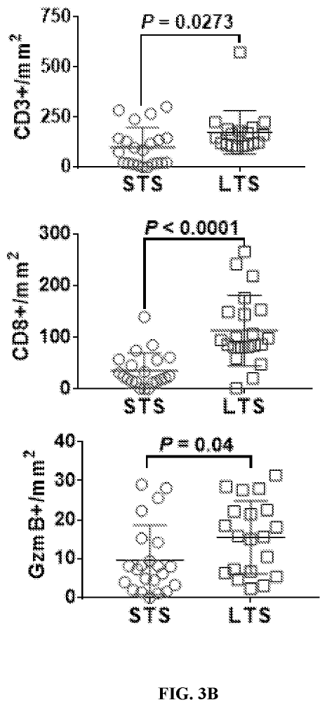

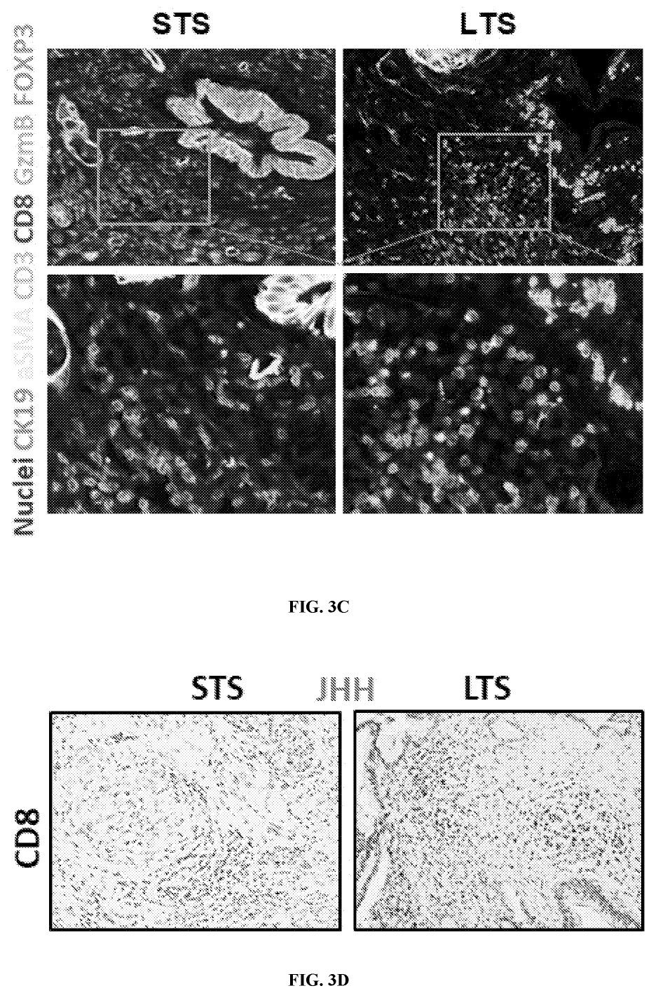

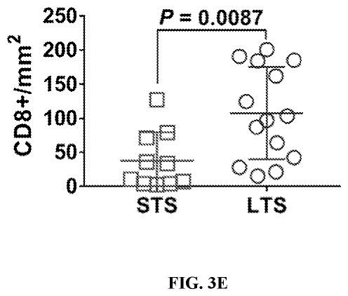

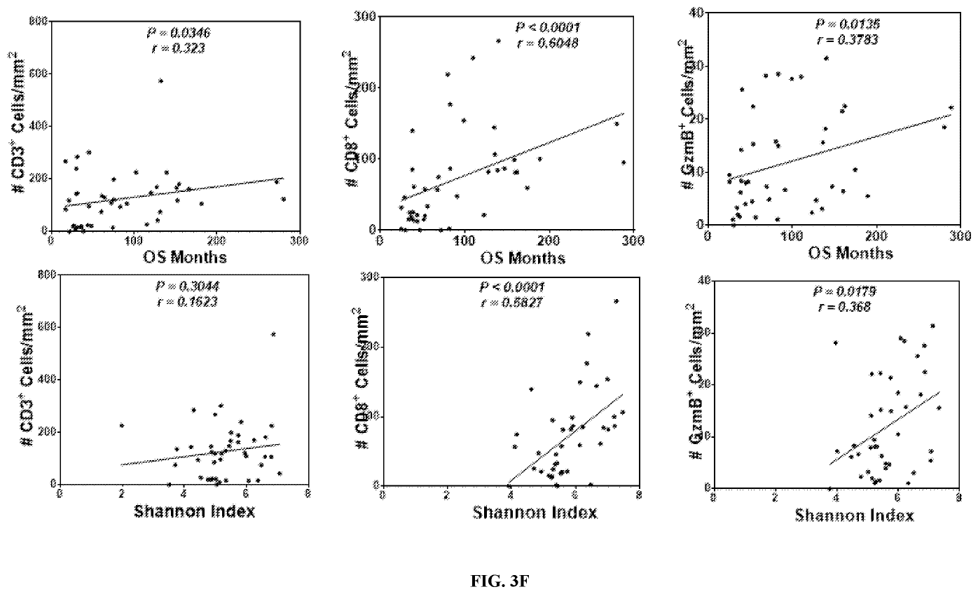

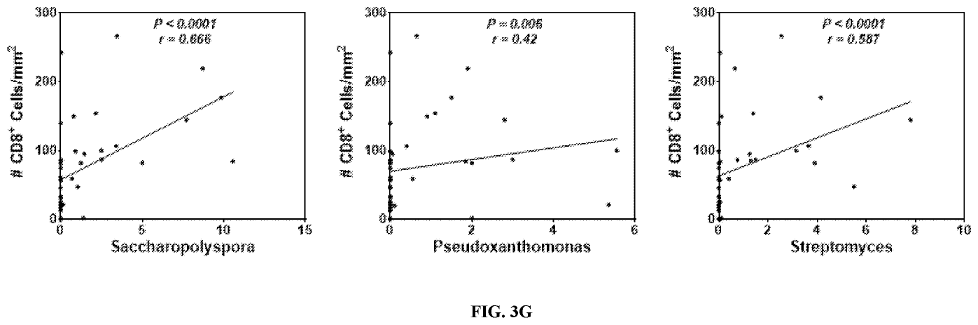

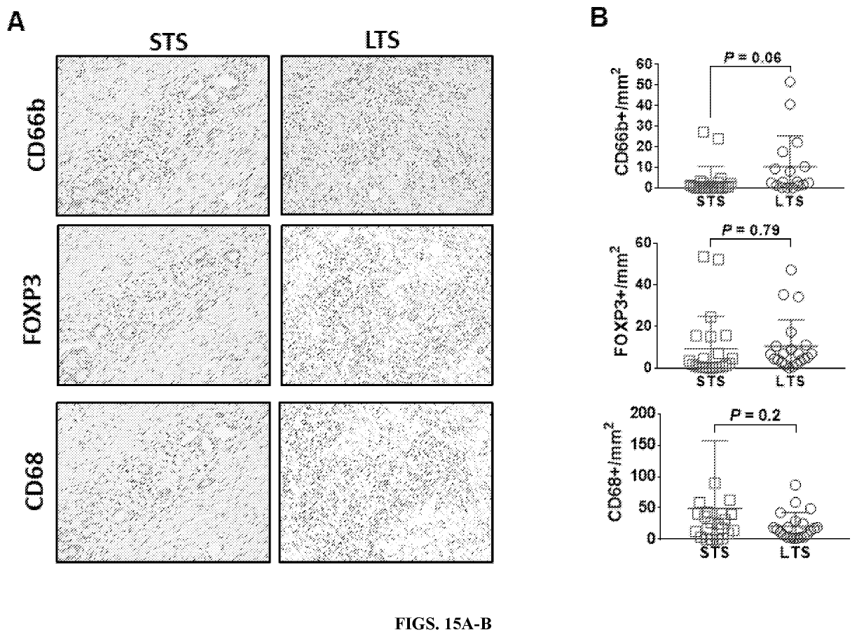

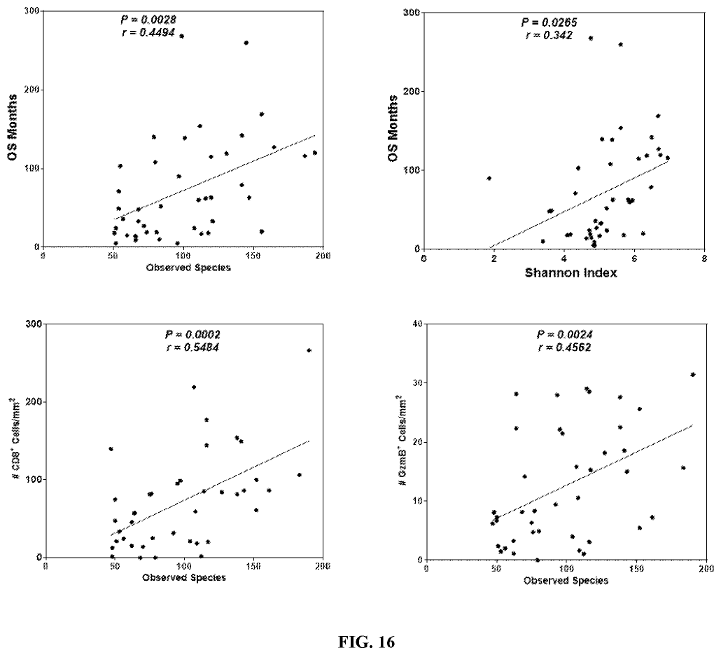

The gut microbiota plays a pivotal role in shaping the immune system (Atarashi et al., 2013; Mazmanian et al., 2008; McAllister et al., 2014). Recent studies have described that the gut microbiota composition can improve responses to immunotherapy by modulating the immune system (Gopalakrishnan et al., 2018; Matson et al., 2018; Riquelme et al., 2018; Routy et al., 2018; Vetizou et al., 2015). It was hypothesized that tumoral bacteria has the ability to shape the immune tumor microenvironment, which can influence the natural history of the cancer. Single-plex immunohistochemistry and multiplex immunofluorescence staining were used to delineate the tumor immune infiltrates in the discovery cohort (MDACC). Greater densities of CD3+ and CD8+ T cells were found in the LTS compared with the STS patients (p=0.0273 and p<0.0001, respectively) (FIGS. 3A-3C). Also, significantly higher numbers of Granzyme B+ cells were detected in the LTS (FIGS. 3A-3B, lower panel, p=0.04), while no significant differences were detected in regulatory T cells (CD3+FOXP3+), macrophages (CD68), or MDSC (CD66b) (FIG. 15). Consistently, greater densities of CD8+ T cells were found in the LTS compared with the STS patients (p=0.008) in the validation cohort (FIGS. 3D&3E). The Spearman rank-order correlation demonstrated a significant positive correlation between CD3+, CD8+, and GzmB+ tissue densities and the overall survival of PDAC patients (p=0.03, p<0.001 and p=0.01, respectively) (FIG. 3F, Table 5). Interestingly, a strong significant correlation was found between CD8+ and GzmB+ tissue densities and microbiome diversity (FIG. 3F, lower panel & FIG. 16), suggesting that the tumor microbiome diversity may influence both the extent of immune infiltration and the degree of activation of CD8+ T cells. Finally, when the CD8+ T cell tissue densities were correlated with the top-three enriched genera in LTS patients, Saccharopolyspora, Pseudoxanthomonas, and Streptomyces, a positive Spearman correlation was found between the two variables (p<0.0001, p=0.006 and p<0.0001, respectively) (FIG. 3G). These finding suggest that the tumor microbiome diversity and the presence of these three genera in the tumor may contribute to the anti-tumor immune response by favoring recruitment and activation of CD8+ T cells.

TABLE 5
Association between survival and immune infiltration
(CD3/CD8 T cells tissue density).

| Variable | HazardRatio | HRLowerCL | HRUpperCL | pValue | Event | Censored | Total |
|---|---|---|---|---|---|---|---|
| CD3 | 0.998 | 0.9941 | 1.002 | 0.3259 | 33 | 10 | 43 |
| CD8 | 0.9894 | 0.9817 | 0.9972 | 0.0076 | 32 | 10 | 42 |

### Example 5—Microbiome Communities from LTS and STS are Associated with Different Metabolic Pathways

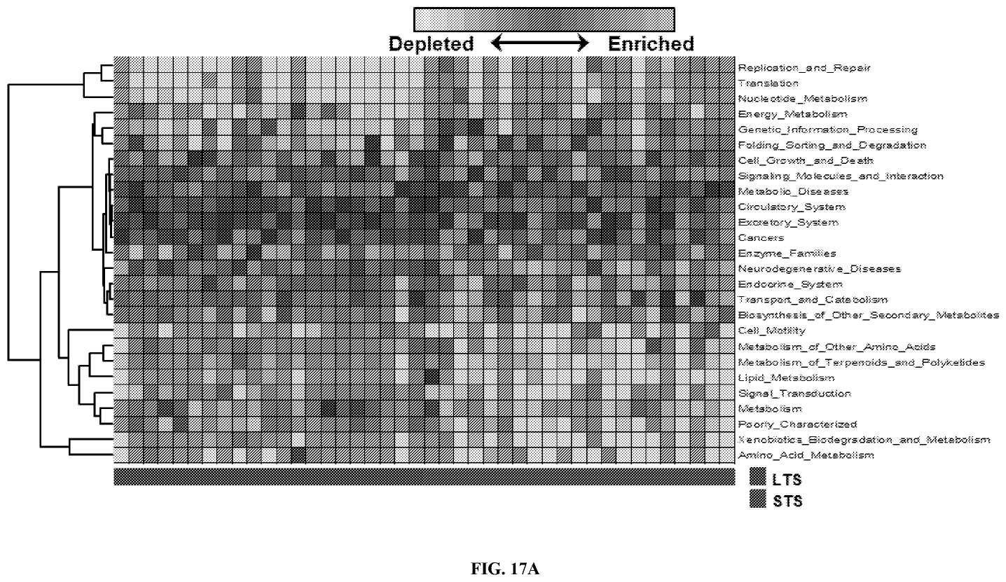

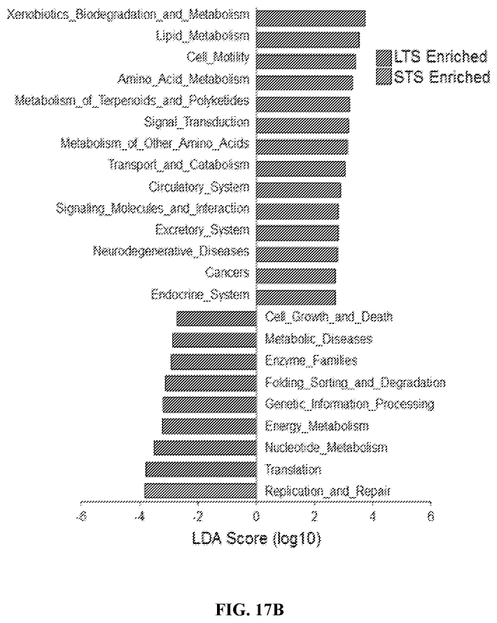

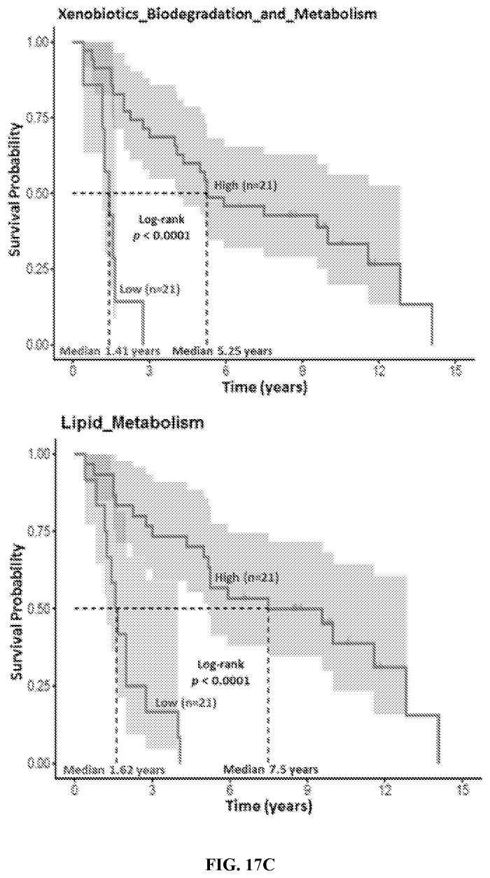

It has been demonstrated that microbiota imbalances can induce systemic metabolic alterations (Devaraj et al., 2013; Nieuwdorp et al., 2014). Conversely, metabolic dysfunction can also induce microbiome imbalances (Cani, 2017). Based on this data, whether the intra-tumoral microbiome is associated with host metabolic pathways was assessed. Phylogenetic Investigation of Communities by Reconstruction of Unobserved States (PICRUSt) (Langille et al., 2013), a technique which uses evolutionary modeling, was used to predict metagenomes from 16S data and reference genome databases (Kanehisa and Goto, 2000). Predicted metagenomes were then used as inputs for metabolic reconstruction using level 2 KEGG Pathways and/or KEGG modules between LTS and STS groups (MDACC cohort; FDR adjusted p<0.05), which were mean-centered and visualized as a heatmap and by Linear Discriminant Analysis (LDA) to assess enrichment and depletion between the two groups. PICRUSt analysis identified 26 core functional modules present across all PDAC samples with a coverage of >90% and p<0.05. Enrichment of differential pathways between LTS and STS groups was detected. Predicted functional categories are involved in important cellular functions and are associated with diverse metabolic and energetic processes (FIGS. 17A&17B). The LTS cases exhibited enrichment in the pathways related to metabolism of amino acids, xenobiotics, lipids, terpenoids and polyketides, besides other cellular functions. Significantly better outcomes were predicted for PDAC patients demonstrating enrichment in xenobiotics biodegradation and lipids metabolism pathways (HR=5.198, 95% CI 1.07-25.06 and HR=4.528, 95% CI 1.54-13.305, respectively) (FIG. 17C). In contrast, the STS cases demonstrated enrichment in synthesis and processing of proteins, processing of genetic information, energetic and nucleotide metabolism, replication, and repair. Some of the pathways shared by both groups, and probably not directly related to the differential outcomes of patients, were pathways related to enzymes, cancer, excretory system, and circulatory system. The PICRUSt taxonomic functional relationships suggest that the composition of the intra-tumoral microbiome determines a differential enrichment of metabolic functional pathways between LTS and STS cases, which may influence patient survival.

### Example 6—Gut Microbiota can Influence Tumor Microbiota and Tumor Growth

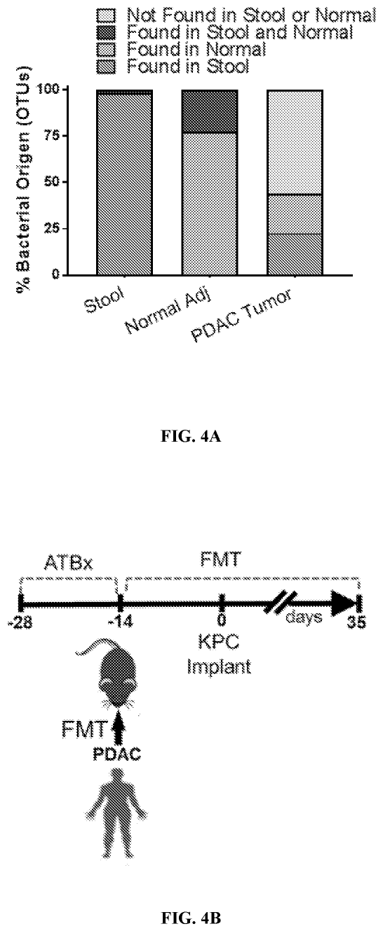

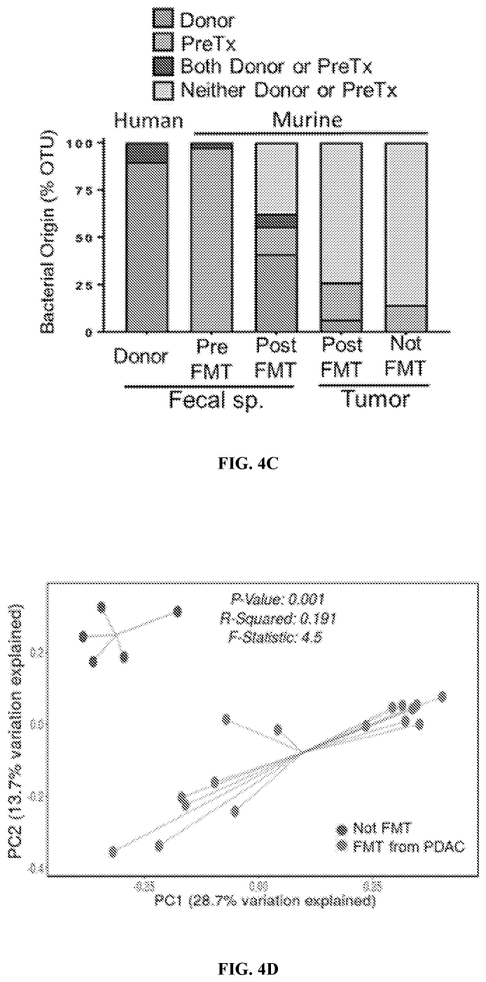

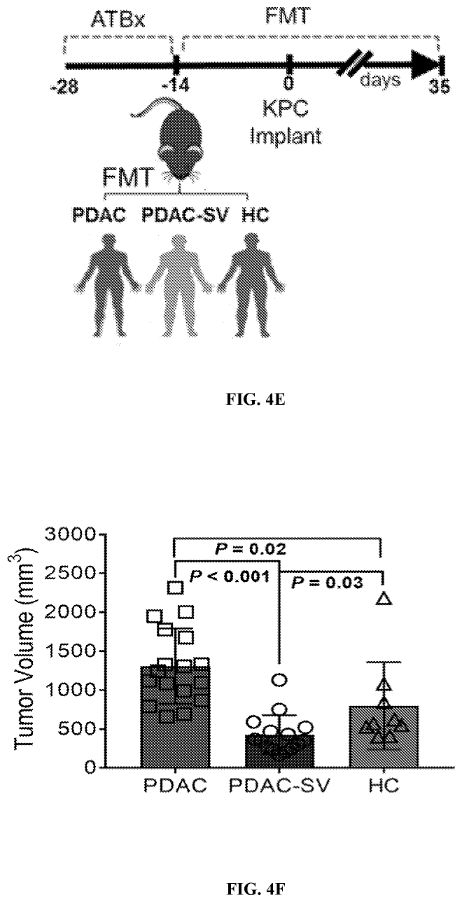

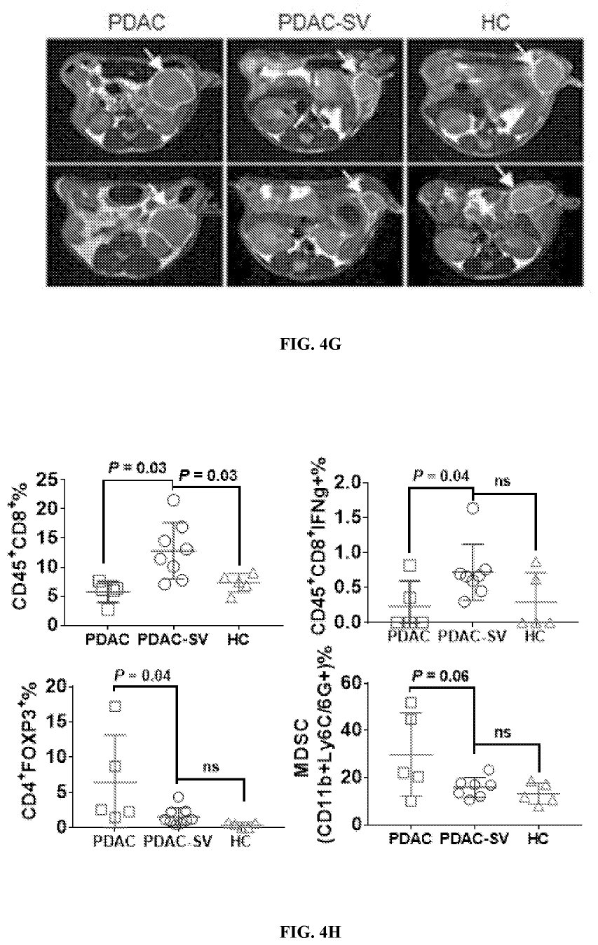

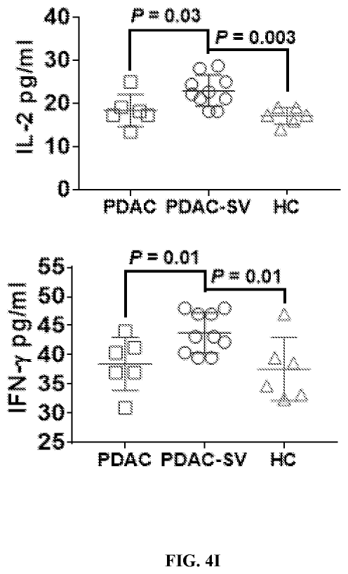

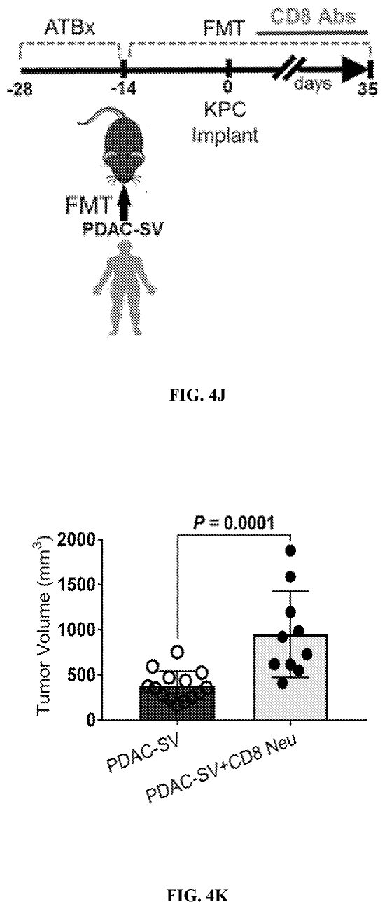

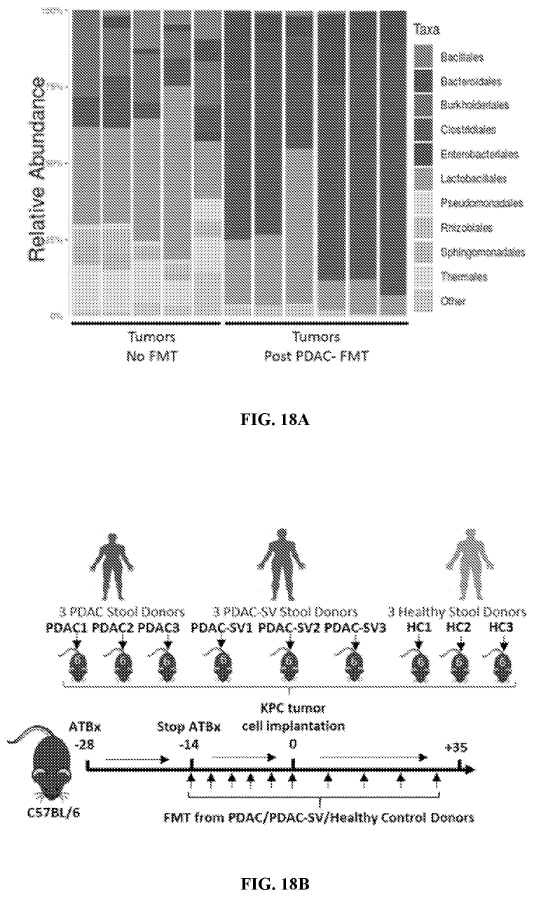

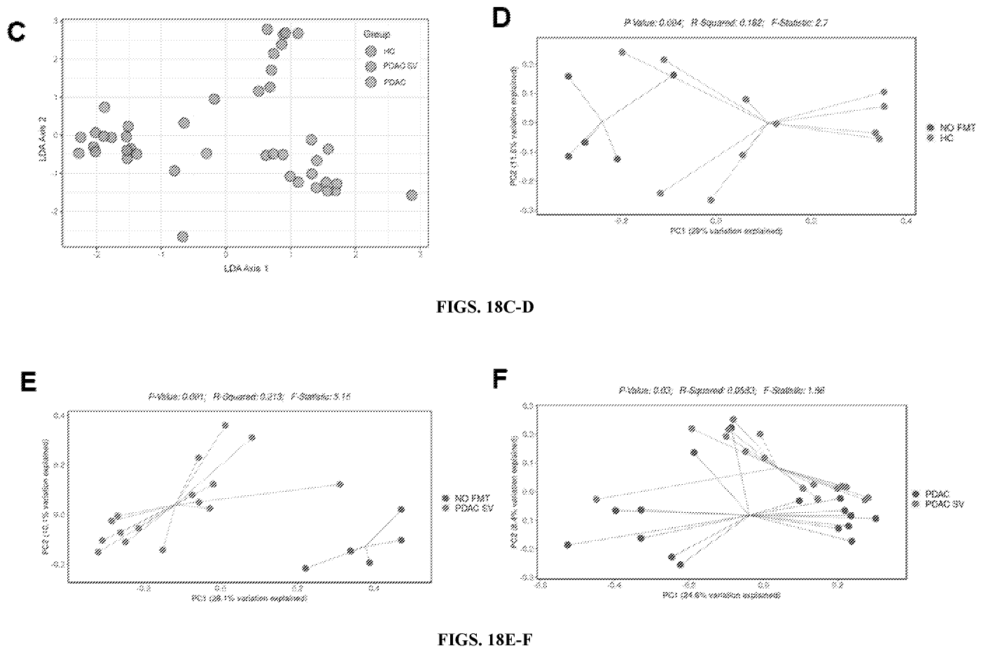

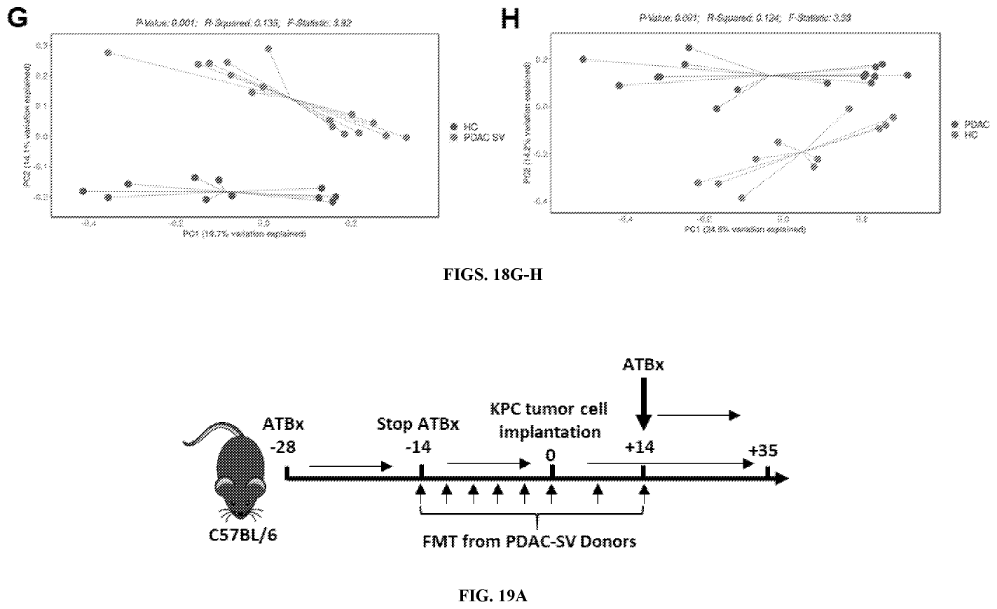

Whether the gut microbiome can modulate the intratumoral microbiome was studied next, and to this end stools, PDAC tumor specimens, and non-tumoral adjacent normal tissue were collected from three patients who underwent Whipple surgery. The taxonomic composition of matched samples was compared and the human gut microbiome was found to represent approximately 25% of the human tumor microbiome, while it is absent from the normal adjacent tissue (FIG. 4A). These data suggest that the gut microbiota has the capacity to specifically colonize pancreatic tumors. To determine if the tumor microbiome could be actively modified by changing the gut microbiome, fecal microbial transplantation (FMT) was performed from patients with advanced PDAC (“STS”) into mice previously treated with antibiotics (ATBx). Human fecal material was transferred by oral gavage three times a week and weekly thereafter. Mice were then challenged with orthotopic implantation of syngeneic cancer lines derived from genetically engineered Pdx1-Cre, LSL-KrasG12D/+, LSL-Trp53R172H/+(“KPC”) mice. The operational taxonomic units (OTUs) abundance in the human PDAC donor samples, murine fecal samples pre-FMT (Basal condition), post-FMT, and at the end point in the murine tumor after 5 weeks of tumor implantation was examined. Interrogation of bacterial origin showed that a large number of bacteria of human donor origin was found as part of the murine gut microbiome (˜40%) of recipient mice post-FMT (FIGS. 4B&4C). Interestingly, human donor bacteria were detected in the murine tumor microbiome post-FMT while it remained absent from mice who did not receive FMT (FIGS. 4B&4C). However, the bacteria coming from donors represented a small percent of the tumor microbiome (<5%), consistent with human data, while the remaining 20% represented the basal murine gut microbiome. Since over 70% of the tumor microbiome was not representative of the gut microbiome (FIG. 4C, grey bars), whether FMT could modulate or shift the overall intratumoral bacterial composition was assessed, in addition to direct translocation. Beta-diversity was used to generate a principal coordinate analysis (PCoA), and a clear differential clustering was detected between OTUs on tumors from mice who received FMT versus mice that did not receive FMT (p<0.001) (FIG. 4D). Additionally, the taxonomic composition of tumors was studied and significant changes in individual bacterial populations were found after FMT (FIG. 18A). Interestingly, one of the bacterial classes that increased in mice who received FMT from STS patient donors was Clostridiales, which was enriched in the original human STS tumor specimens. These data suggest that the gut microbiome can modulate the tumor microbiome, in minor part by direct translocation into the tumors, but more significantly, by altering the microbial landscape.

In order to expand these findings to assess how tumor growth may be affected by differential modulation of the tumor microbiome in response to recipient fecal transplants, stool specimens were obtained from three groups of patients: PDAC patients with advanced disease who would likely experience STS, patients who had PDAC resected more than 5 years prior to collection who would be classified as LTS with no evidence of disease (LTS-NED), and healthy controls (HC). The same FMT approach as described above was employed (FIGS. 4E & 18B). Five weeks after tumor implantation, gut and tumor microbiome and tumor growth were assessed. Gut microbial beta-diversity distinguished the three types of recipient mice (FIG. 18C) and tumor microbial beta-diversity differentially clustered mice groups according to the type of FMT they received (FIGS. 18D-H).

A significant reduction in tumor growth was observed in mice that received FMT from LTS-NED donors compared with the mice transplanted with stools from PDAC patients (p<0.001) or healthy control donors (p=0.02) (FIGS. 4F & 4G). These results suggest that gut/tumor bacteria from patients who had PDAC and survived may have a protective effect against tumors. Tumors from mice who received FMT from STS patients were larger than those from mice who received healthy controls FMT, suggesting that PDAC-associated gut/tumor bacteria may exert a tumor-promoting effect.

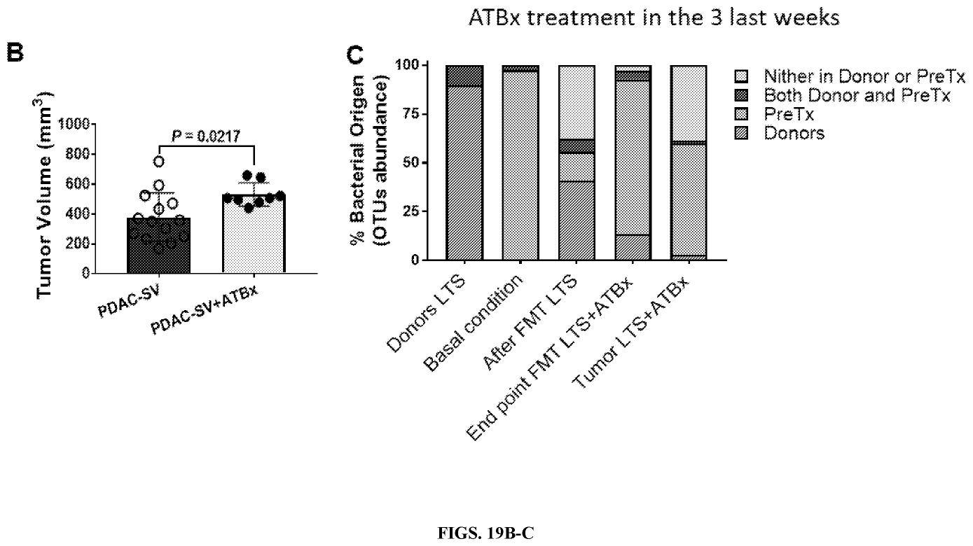

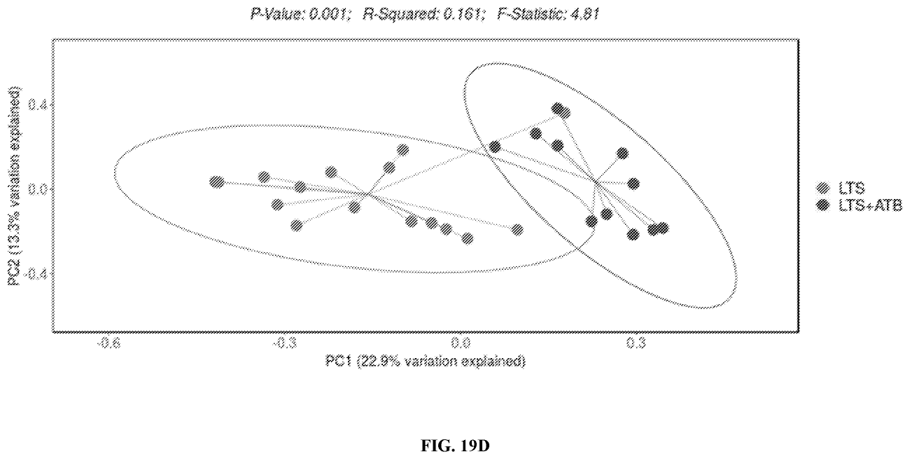

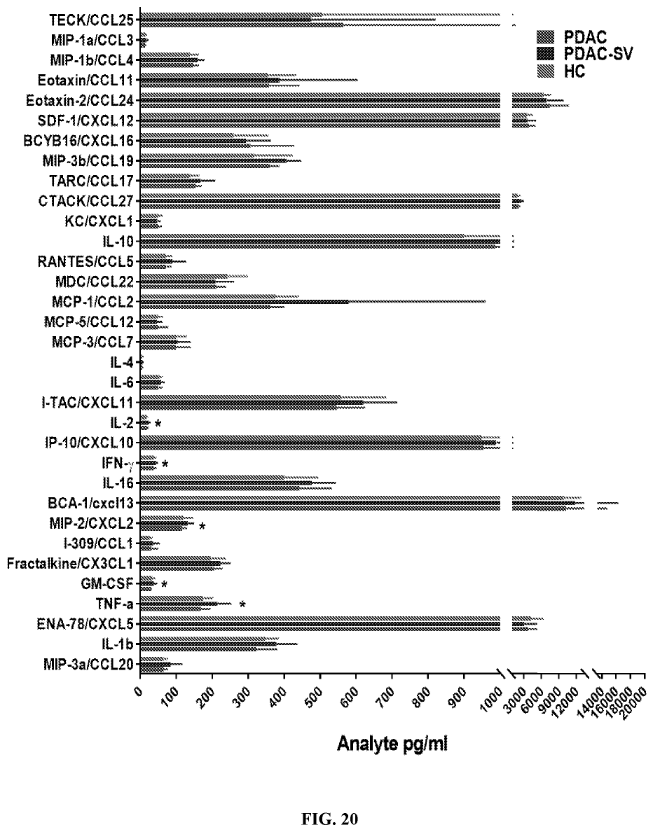

To confirm that the anti-tumoral effect exerted by LTS-NED was definitively induced by changing bacteria content, mice transplanted with stools from LTS-NED were treated with antibiotics post-FMT and compared with mice that did not receive antibiotics post-FMT (FIG. 19A). Short term antibiotics on mice that received FMT from LTS-NED donors induced larger tumors than untreated mice and modified the gut/tumor microbiome (FIGS. 19B & 19C). When the tumor microbiome of the two groups was analyzed, differential clustering for beta-diversity between the two groups was observed (FIG. 19D). These data indicate that bacterial ablation can decrease the anti-tumoral efficacy induced by LTS-NED FMT, which validates the central role of bacteria.

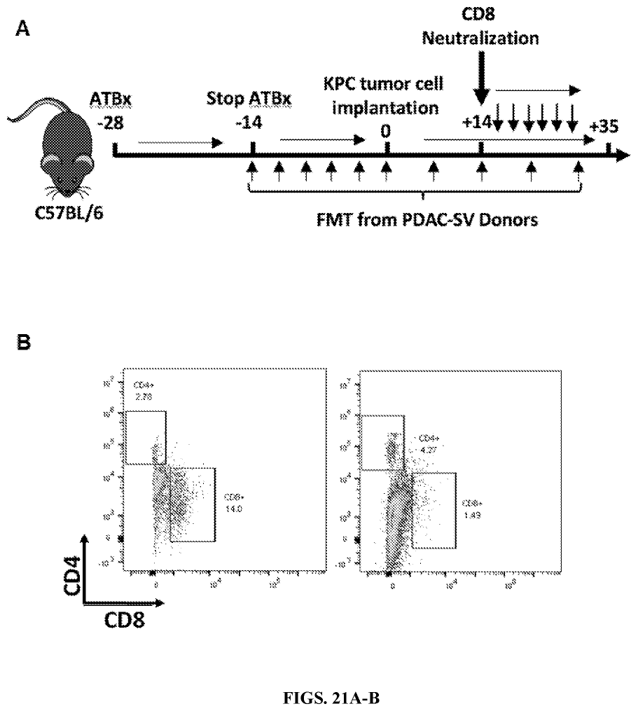

Next, whether the gut microbiome can influence the pancreatic tumor immune infiltrates was assessed. Flow cytometry analysis demonstrated that tumors from mice that received FMT from LTS-NED had significantly higher numbers of CD8+ T cells, as well as CD8+ T cell activation (CD8+/IFNg+ T cells) versus those with stools transferred from PDAC or healthy controls donors, whereas those who received STS FMT had increased CD4+FOXP3+ and myeloid-derived suppressor cells (MDSC) infiltration (FIG. 4H). Additionally, mice receiving FMT from LTS-NED patients had higher serum levels of IFN-γ, IL-2, GM-CSF, TNF-α, and CXCL2 (p<0.05) compared to mice receiving STS FMT (FIGS. 4I & 20). To evaluate the role of T cells in mediating the observed phenotype, CD8+ T cells were depleted using neutralizing antibodies in mice transplanted with PDAC-SV stools, and the mice were subsequently challenged with orthotopic tumors (FIGS. 4J, 21A & 21B). CD8+ T cell depletion blocked the anti-tumoral effect induced by LTS-NED FMT (FIG. 4K), suggesting that the beneficial effect of LTS-NED-associated gut/tumor bacteria is mediated by CD8+ T cells. Together, these data strongly suggest that the gut microbiome can colonize pancreatic tumors, modify its overall tumoral bacterial composition, and modulate immune function to ultimately affect natural history and survival.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Anders & Huber, “Differential expression analysis for sequence count data,” Genome Biol., 11:R106, 2010.
Atarashi et al., “Treg induction by a rationally selected mixture of Clostridia strains from the human microbiota,” Nature, 500:232-236, 2013.
Balachandran et al., “Identification of unique neoantigen qualities in long-term survivors of pancreatic cancer,” Nature, 551:512-516, 2017.
Benjamini et al., “Controlling the false discovery rate in behavior genetics research,” Behav. Brain Res., 125:279-284, 2001.
Bolger et al., “Trimmomatic: a flexible trimmer for Illumina sequence data,” Bioinformatics, 30:2114-2120, 2014.
Cani, “Gut cell metabolism shapes the microbiome,” Science, 357:548-549, 2017.
Caporaso et al., “PyNAST: a flexible tool for aligning sequences to a template alignment,” Bioinformatics, 26:266-267, 2010a.
Caporaso et al., “QIIME allows analysis of high-throughput community sequencing data,” Nat. Methods, 7:335-336, 2010b.
Caporaso et al., “Ultra-high-throughput microbial community analysis on the Illumina HiSeq and MiSeq platforms,” ISME J., 6:1621-1624, 2012.
Dal Molin et al., “Very Long-term Survival Following Resection for Pancreatic Cancer Is Not Explained by Commonly Mutated Genes: Results of Whole-Exome Sequencing Analysis,” Clin. Cancer Res., 21:1944-1950, 2015.
Daquigan et al., “High-resolution profiling of the gut microbiome reveals the extent of Clostridium difficile burden,” NPJ Biofilms Microbiomes, 3:35, 2017.
DeSantis et al., “Cancer treatment and survivorship statistics, 2014,” CA Cancer J. Clin., 64:252-271, 2014.
Devaraj et al., “The human gut microbiome and body metabolism: implications for obesity and diabetes,” Clin. Chem., 59:617-628, 2013.
Drewes et al., “High-resolution bacterial 16S rRNA gene profile meta-analysis and biofilm status reveal common colorectal cancer consortia,” NPJ Biofilms Microbiomes, 3:34, 2017.
Edgar, “Search and clustering orders of magnitude faster than BLAST,” Bioinformatics, 26:2460-2461, 2010.
Edgar et al., “UCHIME improves sensitivity and speed of chimera detection,” Bioinformatics, 27:2194-2200, 2011.
Ferretti et al., “Experimental metagenomics and ribosomal profiling of the human skin microbiome,” Exp. Dermatol., 26:211-219, 2017.
Garrido-Laguna & Hidalgo, “Pancreatic cancer: from state-of-the-art treatments to promising novel therapies,” Nat. Rev. Clin. Oncol., 12:319-334, 2015.
Geller et al., “Potential role of intratumor bacteria in mediating tumor resistance to the chemotherapeutic drug gemcitabine,” Science, 357:1156-1160, 2017.
Gopalakrishnan et al., “Gut microbiome modulates response to anti-PD-1 immunotherapy in melanoma patients,” Science, 359:97-103, 2018.
Heller, “The Cox proportional hazards model with a partly linear relative risk function,” Lifetime Data Anal., 7:255-277, 2001.
Hidalgo, “Pancreatic cancer,” N. Engl. J. Med., 362:1605-1617, 2010.
Human Microbiome Project, “A framework for human microbiome research,” Nature, 486:215-221, 2012a.
Human Microbiome Project, “Structure, function and diversity of the healthy human microbiome,” Nature, 486:207-214, 2012b.
Kamisawa et al., “Pancreatic cancer,” Lancet, 388:73-85, 2016.
Kanehisa & Goto, “KEGG: kyoto encyclopedia of genes and genomes,” Nucleic Acids Res., 28:27-30, 2000.
Kim et al., “Protein kinase D1 is essential for the proinflammatory response induced by hypersensitivity pneumonitis-causing thermophilic actinomycetes Saccharopolyspora rectivirgula,” J Immunol., 184:3145-3156, 2010.
Kuczynski et al., “Using QIIME to analyze 16S rRNA gene sequences from microbial communities,” Curr. Protoc. Bioinformatics, Chapter 10, Unit 10 17, 2011.
Kundu et al., “Our Gut Microbiome: The Evolving Inner Self,” Cell, 171:1481-1493, 2017.
Kurilshikov et al., “Host Genetics and Gut Microbiome: Challenges and Perspectives,” Trends Immunol., 38:633-647, 2017.
Langille et al., “Predictive functional profiling of microbial communities using 16S rRNA marker gene sequences,” Nat. Biotechnol., 31:814-821, 2013.
Langmead & Salzberg, “Fast gapped-read alignment with Bowtie 2,” Nat. Methods, 9:357-359, 2012.
Le et al., “PD-1 Blockade in Tumors with Mismatch-Repair Deficiency,” N. Engl. J. Med., 372:2509-2520, 2015.
Lloyd-Price et al., “Strains, functions and dynamics in the expanded Human Microbiome Project,” Nature, 550:61-66, 2017.
Lozupone et al., “UniFrac: an effective distance metric for microbial community comparison,” ISME J., 5:169-172, 2011.
Magoc & Salzberg, “FLASH: fast length adjustment of short reads to improve genome assemblies,” Bioinformatics, 27:2957-2963, 2011.
Makohon-Moore et al., “Limited heterogeneity of known driver gene mutations among the metastases of individual patients with pancreatic cancer,” Nat. Genet., 49:358-366, 2017.
Manji et al., “Current and Emerging Therapies in Metastatic Pancreatic Cancer,” Clin. Cancer Res., 23:1670-1678, 2017.
Matson et al., “The commensal microbiome is associated with anti-PD-1 efficacy in metastatic melanoma patients,” Science, 359:104-108, 2018.
Mazmanian et al., “A microbial symbiosis factor prevents intestinal inflammatory disease,” Nature, 453:620-625, 453.
McAllister et al., “Microbiota and immune responses in colon cancer: more to learn,” Cancer J., 20:232-236, 2014.
McMurdie & Holmes, “phyloseq: An R Package for Reproducible Interactive Analysis and Graphics of Microbiome Census Data,” PLoS One, 8:e61217, 2013.
Miller et al., “Cancer treatment and survivorship statistics, 2016,” CA Cancer J. Clin., 66:271-289, 2016.
Nieuwdorp et al., “Role of the microbiome in energy regulation and metabolism,” Gastroenterology, 146:1525-1533, 2014.
Ock et al., “Genomic landscape associated with potential response to anti-CTLA-4 treatment in cancers,” Nat. Commun., 8:1050, 2017.
Pushalkar et al., “The Pancreatic Cancer Microbiome Promotes Oncogenesis by Induction of Innate and Adaptive Immune Suppression,” Cancer Discov., 8:403-416, 2018.
Quast et al., “The SILVA ribosomal RNA gene database project: improved data processing and web-based tools,” Nucleic Acids Res., 41:D590-596, 2013.
Riquelme et al., “Immunotherapy for Pancreatic Cancer: More Than Just a Gut Feeling,” Cancer Discov., 8:386-388, 2018.
Routy et al., “Gut microbiome influences efficacy of PD-1-based immunotherapy against epithelial tumors,” Science, 359:91-97, 2018.
Salzman et al., “Enteric defensins are essential regulators of intestinal microbial ecology,” Nat. Immunol., 11:76-83, 2010.
Segata et al., “Metagenomic biomarker discovery and explanation,” Genome Biol., 12:R60, 2011.
Shoemark & Allen, “The microbiome and disease: reviewing the links between the oral microbiome, aging, and Alzheimer's disease,” J. Alzheimers Dis., 43:725-738, 2015.
Siegel et al., “Cancer statistics, 2018,” CA Cancer J. Clin., 68:7-30, 2018.
Vetizou et al., “Anticancer immunotherapy by CTLA-4 blockade relies on the gut microbiota,” Science, 350:1079-1084, 2015.
Vogelstein et al., “Cancer genome landscapes,” Science, 339:1546-1558, 2013.
Wang et al., “Naive Bayesian classifier for rapid assignment of rRNA sequences into the new bacterial taxonomy,” Appl. Environ. Microbiol., 73:5261-5267, 2007.

What is claimed is:

1. A method of classifying a patient having pancreatic ductal adenocarcinoma as being either a short-term survivor or a long-term survivor, the method comprising:
(a) obtaining a sample of the patient's tumor;
(b) detecting the presence of at least three bacterial species in the sample; and
(c) classifying the patient having pancreatic ductal adenocarcinoma as being either a short-term survivor or a long-term survivor based on the bacterial species detected.

2. The method of claim 1, wherein if the bacterial species detected belong to the Alphaprotebacteria, Sphingobacteria, and/or Flavobacteria class, then the patient is classified as being a long-term survivor.

3. The method of claim 1, wherein if the bacterial species detected belong to the Clostridia and/or Bacteroides class, then the patient is classified as being a short-term survivor.

4. The method of claim 1, wherein if the bacterial species detected belong to the Proteobacteria and/or Actinobacteria genus, then the patient is classified as being a long-term survivor.

5. The method of claim 1, wherein if the bacterial species detected belong to the Pseudoxanthomonas, Streptomyces, Bacillus, and/or Saccharopolyspora taxon, then the patient is classified as being a long-term survivor.

6. The method of claim 1, wherein step (c) further comprises determining an alpha diversity level based on the bacterial species detected.

7. The method of claim 6, wherein if the alpha diversity level is higher than a reference level, then the patient is classified as being a long-term survivor.

8. The method of claim 7, wherein the reference level is an alpha diversity level in a healthy pancreas.

9. The method of any one of claims 1-6, wherein the sample is a formalin-fixed, paraffin-embedded sample.

10. The method of any one of claims 1-6, wherein the sample is a fresh frozen sample.

11. The method of any one of claims 1-10, further comprising reporting the classification of the patient.

12. The method of claim 11, wherein the reporting comprises preparing a written or electronic report.

13. The method of claim 12, further comprising providing the report to the patient, a doctor, a hospital, or an insurance company.

14. The method of claim 1, wherein if the patient is classified as being a long-term survivor, then the method further comprises performing Whipple surgery on the patient, administering chemotherapy to the patient, and/or administering radiation therapy to the patient.

15. The method of claim 1, wherein if the patient is classified as being a short-term survivor, then the method further comprises performing Whipple surgery on the patient, administering chemotherapy to the patient, and/or administering radiation therapy to the patient.

16. The method of claim 1, wherein if the patient is classified as being a short-term survivor, then the method further comprises performing Fecal Microbiota Transplantation (FMT) from a survivor of pancreatic ductal adenocarcinoma to the patient.

17. The method of claim 16, wherein the survivor of pancreatic ductal adenocarcinoma is in remission.

18. The method of claim 17, wherein the survivor of pancreatic ductal adenocarcinoma has been in remission for at least five years.

19. The method of claim 16, wherein the patient is treated with antibiotics prior to the FMT.

20. The method of claim 16, wherein the patient is not treated with antibiotics prior to the FMT.

21. A method of treating a patient having pancreatic ductal adenocarcinoma, the method comprising performing Fecal Microbiota Transplantation (FMT) from a survivor of pancreatic ductal adenocarcinoma to the patient.

22. The method of claim 21, wherein the patient is treated with antibiotics prior to the FMT.

23. The method of claim 21, wherein the patient is not treated with antibiotics prior to the FMT.

24. The method of any one of claims 21-23, further comprising performing Whipple surgery on the patient.

25. The method of any one of claims 21-24, wherein the survivor of pancreatic ductal adenocarcinoma is in remission.

26. The method of any one of claims 21-25, wherein the survivor of pancreatic ductal adenocarcinoma has been in remission for at least five years.

27. A method for inducing intra-tumoral immune cell infiltration in a patient having pancreatic ductal adenocarcinoma, the method comprising performing Fecal Microbiota Transplantation (FMT) from a survivor of pancreatic ductal adenocarcinoma to the patient.

28. The method of claim 27, wherein the patient is treated with antibiotics prior to the FMT.

29. The method of claim 27, wherein the patient is not treated with antibiotics prior to the FMT.

30. The method of any one of claims 27-29, wherein the survivor of pancreatic ductal adenocarcinoma is in remission.

31. The method of any one of claims 27-30, wherein the survivor of pancreatic ductal adenocarcinoma has been in remission for at least five years.

32. The method of any one of claims 27-31, wherein the method induces the infiltration of CD8+ T cells.

33. A method for decreasing tumor infiltration by Tregs in a patient having pancreatic ductal adenocarcinoma, the method comprising performing Fecal Microbiota Transplantation (FMT) from a survivor of pancreatic ductal adenocarcinoma or a healthy subject to the patient.

34. The method of claim 33, wherein the patient is treated with antibiotics prior to the FMT.

35. The method of claim 33, wherein the patient is not treated with antibiotics prior to the FMT.

36. The method of any one of claims 33-35, wherein the survivor of pancreatic ductal adenocarcinoma is in remission.

37. The method of any one of claims 33-36 wherein the survivor of pancreatic ductal adenocarcinoma has been in remission for at least five years.

38. A pharmaceutical composition comprising fecal microbiota obtained from a survivor of pancreatic ductal adenocarcinoma and a pharmaceutically acceptable carrier.

39. The composition of claim 38, wherein the survivor of pancreatic ductal adenocarcinoma is in remission.

40. The composition of claim 38, wherein the survivor of pancreatic ductal adenocarcinoma has been in remission for at least five years.

41. A method for inducing an immunoactive microenvironment in a pancreatic ductal adenocarcinoma having an immunosuppressive microenvironment, the method comprising performing Fecal Microbiota Transplantation (FMT) from a survivor of pancreatic ductal adenocarcinoma or a healthy subject to a patient having a pancreatic ductal adenocarcinoma with an immunosuppressive microenvironment.

42. The method of claim 41, comprising performing Fecal Microbiota Transplantation (FMT) from a survivor of pancreatic ductal adenocarcinoma to the patient.

43. The method of claim 41 or 42, wherein the patient is treated with antibiotics prior to the FMT.

44. The method of claim 41 or 42, wherein the patient is not treated with antibiotics prior to the FMT.

45. The method of any one of claims 41-44, wherein the survivor of pancreatic ductal adenocarcinoma is in remission.

46. The method of any one of claims 41-45, wherein the survivor of pancreatic ductal adenocarcinoma has been in remission for at least five years.

47. A method of sensitizing a pancreatic ductal adenocarcinoma having an immunosuppressive microenvironment to immune checkpoint inhibitors, the method comprising performing Fecal Microbiota Transplantation (FMT) from a survivor of pancreatic ductal adenocarcinoma or a healthy subject to a patient having a pancreatic ductal adenocarcinoma with an immunosuppressive microenvironment.

48. The method of claim 47, comprising performing Fecal Microbiota Transplantation (FMT) from a survivor of pancreatic ductal adenocarcinoma to the patient.

49. The method of claim 47 or 48, wherein the patient is treated with antibiotics prior to the FMT.

50. The method of claim 47 or 48, wherein the patient is not treated with antibiotics prior to the FMT.

51. The method of any one of claims 47-50, wherein the survivor of pancreatic ductal adenocarcinoma is in remission.

52. The method of any one of claims 47-51, wherein the survivor of pancreatic ductal adenocarcinoma has been in remission for at least five years.

53. The method of any one of claims 47-52, further comprising administering an immune checkpoint inhibitor to the patient.

54. A method of treating a patient having pancreatic ductal adenocarcinoma, the method comprising (1) performing Fecal Microbiota Transplantation (FMT) from a survivor of pancreatic ductal adenocarcinoma or a healthy subject to the patient and (2) administering an immune checkpoint inhibitor to the patient.

55. The method of claim 54, comprising performing Fecal Microbiota Transplantation (FMT) from a survivor of pancreatic ductal adenocarcinoma to the patient.

56. The method of claim 54 or 55, wherein the patient is treated with antibiotics prior to the FMT.

57. The method of claim 54 or 55, wherein the patient is not treated with antibiotics prior to the FMT.

58. The method of any one of claims 54-57, further comprising performing Whipple surgery on the patient.

59. The method of any one of claims 54-58, wherein the survivor of pancreatic ductal adenocarcinoma is in remission.

60. The method of any one of claims 54-59, wherein the survivor of pancreatic ductal adenocarcinoma has been in remission for at least five years.

61. The method of any one of claims 54-60, wherein the immune checkpoint inhibitor targets adenosine A2A receptor (A2AR), B7-H3 (also known as CD276), B and T lymphocyte attenuator (BTLA), cytotoxic T-lymphocyte-associated protein 4 (CTLA-4, also known as CD152), glucocorticoid-induced tumour necrosis factor receptor-related protein (GITR), indoleamine 2,3-dioxygenase (IDO), killer-cell immunoglobulin (KIR), lymphocyte activation gene-3 (LAG3), Mer tyrosine kinase (MerTK), OX40, programmed death 1 (PD-1), programmed death-ligand 1 (PD-L1), T cell immunoreceptor with Ig and ITIM domains (TIGIT), T-cell immunoglobulin domain and mucin domain 3 (TIM-3), or V-domain Ig suppressor of T cell activation (VISTA).

62. The method of any one of claims 54-61, wherein the immune checkpoint inhibitor comprises one or more of an anti-PD1 therapy, an anti-PD-L1 therapy, and an anti-CTLA-4 therapy.
